(19) United States
(12) Patent Application Publication
(10) Pub. No.: US 2025/0375511 A1
(43) Pub. Date: Dec. 11, 2025

(54) METHOD FOR TREATING CONDITIONS ASSOCIATED WITH HYPERPROLIFERATING CELLS COMPRISING COMBINED ADMINISTRATION OF INFECTIOUS AGENTS AND METAL CATIONS

(71) Applicant: Theralase Technologies, Inc., Toronto (CA)

(72) Inventors: Arkady MANDEL, North York (CA); Roger Dumoulin-White, Toronto (CA)

(21) Appl. No.: 18/734,679

(22) Filed: Jun. 5, 2024

(51) Int. Cl. A61K 39/04; A61K 39/00; A61K 47/54; A61P 35/00

(52) U.S. Cl. CPC A61K 39/04; A61K 47/545; A61P 35/00; A61K 2039/522; A61K 2039/572; A61K 2039/585; A61K 2039/80

(57) ABSTRACT

Disclosed is a composition for treating a condition associated with hyperproliferation of cells, including at least one infectious agent selected from the group consisting of bacteria, protozoa, fungi, viruses, algae and structural components thereof, wherein the at least one infectious agent has a net negative charge and is effective to treat the condition; and at least one metal cation in an amount effective to more than offset the net negative charge of the at least one infectious agent such that the composition has a net positive charge. Also disclosed is a method for treating a condition associated with hyperproliferation of cells in a patient, including administering to the patient at least one infectious agent as described above and administering to the patient at least one metal cation as described above, wherein the method is effective to slow cell hyperproliferation, slow tumor growth and/or reduce tumor volume.

### BACKGROUND OF THE INVENTION

### 1. Field of Invention

This invention relates to cancer immunotherapy using infectious agents, and to metal complexes and methods for their use in the prevention or treatment of cancer.

### 2. Description of Related Art

Bacteria-based cancer immunotherapy uses modified bacteria to specifically target and colonize tumor tissues, triggering anti-tumor immune responses and eliminating tumor cells. See, e.g., Fan et al., 2022.

Bacillus Calmette-Guerin (BCG) is an attenuated form of Mycobacterium bovis and, besides its historic use as vaccine against tuberculosis, BCG has proved to be effective as anti-cancer treatment for urinary bladder cancer and potentially other types of cancer (Han, 2020). BCG is currently used to treat patients with some low-grade and high-grade non-muscle invasive bladder cancer (NMIBC).

As typical for a Gram-positive bacterium, the BCG cell wall has a strong negative charge, largely due to phosphate-rich teichoic acid in the cell wall (Petit et al., 1975), as well as sulphate and carboxylic groups. This negative charge may persist independently of the environment pH, and both in live and killed BCG cells. In some BCG strains, loosely adherent surface proteins contribute to the negative charge, which in this case is pH-dependent (requires pH>3.0-4.4) (Zhang et al., 1988; Shi et al., 1989; Kristensen et al., 1992). Combination of the highly negatively charge of both the urothelium (and especially tumor cells surface) and the BCG cells forces the latter ones to accumulate in a very close proximity of the bladder wall (7-10 nm) (Schamhart et al., 1994) and makes irreversible adherence of BCG to the urothelium/tumor difficult and infrequent (Ratliff et al., 1987; Hudson et al., 1991; Teppema et al., 1992). However, when the attachment and uptake of BCG bacteria happens, they can be detected in urothelial cells as early as after 15 minutes of incubation and can penetrate several cell layers deep in bladder tumor model; this does not happen in normal urothelial cells (Durek et al., 1999). Poorly differentiated (and more aggressive) bladder cancer cells internalize BCG better than well-differentiated cells (Bevers et al, 1998); this is in accordance with better responsiveness of high-grade bladder cancer tumors to BCG vs. that of low-grade ones (Melekos et al., 1993). Nevertheless, the electrostatic barrier between the BCG cells and bladder cancer cells necessitates multiple sessions of administration of high BCG doses for anti-cancer intravesical therapy (Poggi et al., 2022). Any approach introducing more positively charged anti-cancer agent is therefore beneficial; conjugation of drugs to polyethylene glycol is an example (Lele & Hoffman, 2000; Guo et al., 2020). In this respect, reversion of zeta potential of BCG cells to +60 mV (from intrinsic −40 mV) by addition of cationic surfactant cetylpyridinium chloride (Zhang et al., 1988) is a promising approach in increasing anti-cancer formulations efficacy and safety (due to less prolonged and lower dose administration without sacrificing efficacy).

Most patients that receive BCG experience tumor recurrence over time, and many patients can develop high-grade NMIBC that is BCG-unresponsive.

Radical cystectomy is another currently available treatment for high-grade NMIBC; however, the procedure is associated with high perioperative morbidity, and many patients are unwilling or unable to undergo the procedure. Various agents have been evaluated as salvage intravesical therapies after treatment with BCG; however, none to date have provided robust and durable responses in patients.

Thus, there is an unmet need for effective and safe treatments for patients with bladder cancers, particularly high-grade NMIBC.

Moreover, it is desired to identify means for expanding the use of infectious agents suitable for cancer therapy, including BCG, in local and/or systemic cancer therapy.

Glycosaminoglycans (mucopolysacharides) are highly sulphated molecules and involve chondroitin sulfate, hyaluronic acid, keratan sulfate and heparin/heparan sulfate, (HSGAGs). They are negatively charged and create a thick hydrophilic mucus layer on the surface of the urothelial umbrella cells. Glycosaminoglycans serve as barrier against various potentially aggressive compounds in urine, as well as bacterial infection preventing thus inflammatory diseases in the bladder. Moreover, glycosaminoglycans are present not only on the surface but in deeper layers of urinary bladder wall including stroma (Poggi et al., 2000; Bevers et al., 2004; Davis et al., 2006; Hurst et al., 2007; Costantini et al., 2013). Glycosaminoglycans also play a role in epithelial differentiation by regulating interactions between epithelium and stroma and by modulation of cytokines release (Hurst et al., 1994). This barrier is further facilitated on cellular level in the bladder malignant tumors. Bladder cancer cells have stronger negative surface charge because of greater glycosylation and phospholipids content than in normal urothelial cells (von Palubitzki et al., 2020) and more acidic (pH=6.2-6.9) tumor microenvironment compared to pH=7.3-7.4 in normal tissue (Cardone et al., 2005; Reshkin et al., 2013). Negatively charged hyaluronidase is an established urinary marker for bladder cancer (Nakamura et al., 2014; Dobrzyńska et al., 2015).

All references cited herein are incorporated herein by reference in their entireties. The citation of any reference is not to be construed as an admission that it is prior art with respect to the present invention.

### BRIEF SUMMARY OF THE INVENTION

Accordingly, a first aspect of the invention is a composition for treating a condition associated with hyperproliferation of cells, comprising: at least one infectious agent selected from the group consisting of bacteria, protozoa, fungi, viruses, algae and structural components thereof, wherein the at least one infectious agent has a net negative charge and is effective to treat the condition; and at least one metal cation in an amount effective to more than offset the net negative charge of the at least one infectious agent such that the composition has a net positive charge.

In certain embodiments, the at least one metal cation is contained in at least one metal complex represented by formula (I):

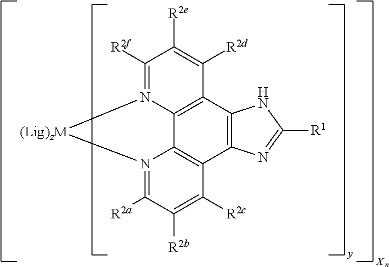

including hydrates, solvates, pharmaceutically acceptable salts and prodrugs thereof, wherein:
the at least one metal cation (M) is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
y=1, 2, or 3;
z=0, 1, or 2;
Lig at each occurrence is independently selected from the group consisting of

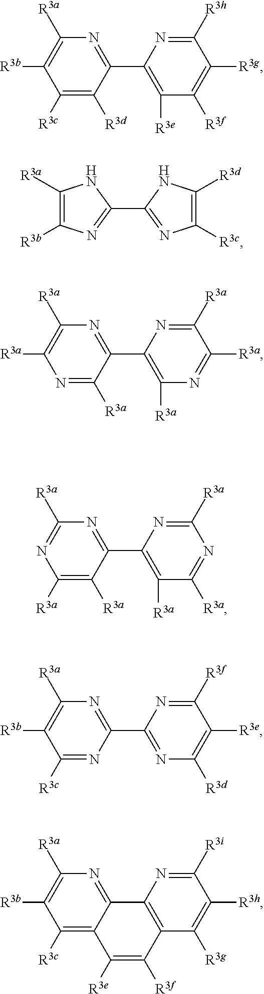

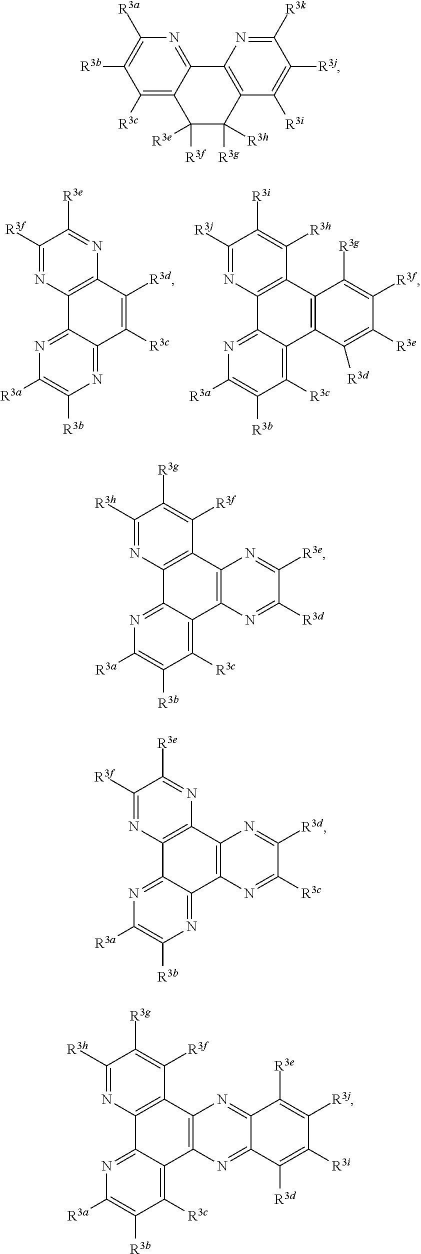

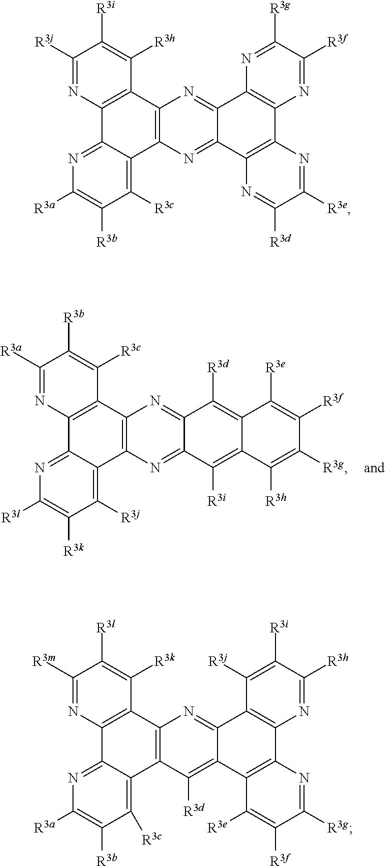

R¹ is selected from the group consisting of

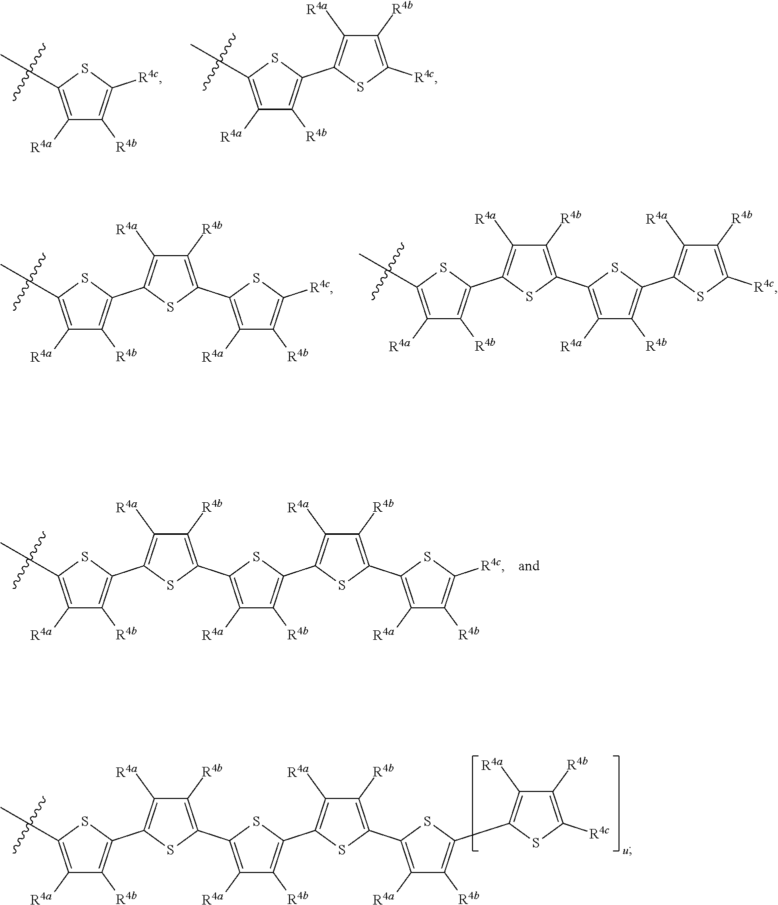

u is an integer of 1 to 20;
R^{2a}, R^{2b}, R^{2c}, R^{2d}, R^{2e}, and R^{2f} at each occurrence are each independently selected from the group consisting of hydrogen, C1-6 optionally substituted alkyl, C1-6 optionally substituted branched alkyl, C3-7 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h}, R³ⁱ, R^{3j}, R^{3k}, R^{3l}, and R^{3m} at each occurrence are each independently selected from the group consisting of hydrogen, C1-6 optionally substituted alkyl, C1-6 optionally substituted branched alkyl, C1-6 optionally substituted haloalkyl, C1-6 optionally substituted alkoxy, and CO₂R¹;
R^{4a}, R^{4b}, and R^{4c} at each occurrence are each independently selected from the group consisting of hydrogen, C1-6 optionally substituted alkyl, C1-6 optionally substituted branched alkyl, C1-6 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;
R⁶ at each occurrence is independently selected from the group consisting of hydrogen and optionally substituted alkyl;
R⁷ at each occurrence is independently selected from the group consisting of hydrogen and optionally substituted alkyl; and
R⁸ at each occurrence is independently selected from the group consisting of hydrogen and optionally substituted alkyl.

In certain embodiments, the composition comprises complexes of the at least one infectious agent and the at least one metal complex.

In certain embodiments, the composition further comprises transferrin.

In certain embodiments, the at least one metal complex comprises at least one member selected from the group consisting of:
Ru(2,2′-bipyridine)₂(2-(2′,2″:5″,2′″-terthiophene)-imidazo[4,5-]A[1,10]phenanthroline);
Ru(2,2′-bipyridine)₂(2-(2′,2″:5″,2′″;5′″,2″″-quaterthiophene)-imidazo[4,5-f][1,10]phenanthroline);
Ru(4,4′-dimethyl-2,2′-bipyridine)₂(2-thiophenimidazo[4,5-f][1,10]phenanthroline);
Ru(4,4′-dimethyl-2,2′-bipyridine)₂(2-(2′,2″-bithiophene)-imidazo[4,5-f][1,10]phenanthroline);
Ru(4,4′-dimethyl-2,2′-bipyridine)₂(2-(2′,2″:5″,2′″-terthiophene)-imidazo[4,5-f][1,10]phenanthroline);
Ru(4,4′-dimethyl-2,2′-bipyridine)₂(2-(2′,2′″:5″,2′″;5′″,2″″-quaterthiophene)-imidazo[4,5-f][1,10]phenanthroline);
Ru(4,4′-di-t-butyl-2,2′-bipyridine)₂(2-thiophenimidazo[4,5-f][1,10]phenanthroline);
Ru(4,4′-di-t-butyl-2,2′-bipyridine)₂(2-(2′,2″-bithiophene)-imidazo[4,5-f][1,10]phenanthroline);
Ru(4,4′-di-t-butyl-2,2′-bipyridine)₂(2-(2′,2″:5″,2′″-terthiophene)-imidazo[4,5-f][1,10]phenanthroline);
Ru(4,4′-dimethoxy-2,2′-bipyridine)₂(2-(2′,2″:5″,2′″-terthiophene)-imidazo[4,5-f][1,10]phenanthroline);
Ru(5,5′-dimethyl-2,2′-bipyridine)₂(2-thiophenimidazo[4,5-f][1,10]phenanthroline);
Ru(5,5′-dimethyl-2,2′-bipyridine)₂(2-(2′,2″-bithiophene)-imidazo[4,5-f][1,10]phenanthroline);
Ru(5,5′-dimethyl-2,2′-bipyridine)₂(2-(2′,2″:5″,2′″-terthiophene)-imidazo[4,5-f][1,10]phenanthroline);
Ru(6,6′-dimethyl-2,2′-bipyridine)₂(2-thiophenimidazo[4,5-f][1,10]phenanthroline);
Ru(6,6′-dimethyl-2,2′-bipyridine)₂(2-(2′,2″-bithiophene)-imidazo[4,5-f][1,10]phenanthroline);
Ru(6,6′-dimethyl-2,2′-bipyridine)₂(2-(2′,2″:5″,2′″-terthiophene)-imidazo[4,5-f][1,10]phenanthroline);
Ru(4,4′-di(methylcarboxy)-2,2′-bipyridine)₂(2-(2′,2′″:5″,2′″-terthiophene)-imidazo[4,5-f][1,10]phenanthroline);
Ru(2,2′-bipyrimidine)₂(2-(2′,2″:5″,2′″-terthiophene)-imidazo[4,5-f][1,10]phenanthroline);
Ru(2,2′-bipyrimidine)(4,4′-dimethyl-2,2′-bipyridine)(2-(2′,2′″:5″,2′″-terthiophene)-imidazo[4,5-f][1,10]phenanthroline);
Ru(1,10-phenanthroline)₂(2-thiophenimidazo[4,5-f][1,10]phenanthroline);
Os(2,2′-bipyridine)₂(2-thiophenimidazo[4,5-f][1,10]phenanthroline);
Os(2,2′-bipyridine)₂(2-(2′,2″-bithiophene)-imidazo[4,5-f][1,10]phenanthroline);
Os(2,2′-bipyridine)₂(2-(2′,2″:5″,2′″-terthiophene)-imidazo[4,5-f][1,10]phenanthroline);
Os(1,10-phenanthroline)₂(2-thiophenimidazo[4,5-f][1,10]phenanthroline);
Os(1,10-phenanthroline)₂(2-(2′,2″-bithiophene)-imidazo[4,5-][1,10]phenanthroline);
Os(1,10-phenanthroline)₂(2-(2′,2″:5″,2′″-terthiophene)-imidazo[4,5-][1,10]phenanthroline);
Os(4,4′-dimethyl-2,2′-bipyridine)₂(2-thiophenimidazo[4,5-f][1,10]phenanthroline);
Os(4,4′-dimethyl-2,2′-bipyridine)₂(2-(2′,2″-bithiophene)-imidazo[4,5-f][1,10]phenanthroline);
Os(4,4′-dimethyl-2,2′-bipyridine)₂(2-(2′,2′″:5″,2′″-terthiophene)-imidazo[4,5-f][1,10]phenanthroline); and
pharmaceutically acceptable salts thereof.

In certain embodiments, the at least one metal complex is Ru(4,4′-dimethyl-2,2′-bipyridine)₂(2-(2′,2″:5″,2′″-terthiophene)-imidazo[4,5-f][1,10]phenanthroline) or a pharmaceutically acceptable salt thereof.

In certain embodiments, a single dose of the composition contains 0.001 μg to 80000 μg of the at least one metal complex and 1×10⁻⁴ to 8×10⁻⁸ CFU of the at least one infectious agent.

In certain embodiments, the composition further comprises at least one negatively charged substance additional to the at least one infectious agent, wherein the negatively charged substance is at least one of a lipopolysaccharide and a negatively charged microorganism.

In certain embodiments, the at least one infectious agent comprises engineered bacteria selected from the group consisting of live attenuated Mycobacterium Bovis, engineered Salmonella, engineered Staphylococcus, engineered Listeria, engineered E-coli, engineered Bifidobacterium and engineered Clostridium.

In certain embodiments, the composition comprises at least two different infectious agents selected from the group consisting of bacteria, protozoa, fungi, viruses, algae and structural components thereof.

A second aspect of the invention is a method for treating a condition associated with hyperproliferation of cells in a patient, comprising the steps:
(a) administering to the patient at least one infectious agent selected from the group consisting of bacteria, protozoa, fungi, viruses and algae, wherein the at least one infectious agent has a net negative charge and is effective to treat the condition; and
(b) administering to the patient before, during and/or after step (a) at least one metal cation,
wherein the method is effective to slow cell hyperproliferation, slow tumor growth and/or reduce tumor volume.

A third aspect of the invention is a method for treating a condition associated with hyperproliferation of cells in a patient, comprising administering to the patient a composition of the invention in an amount and for a duration effective to slow cell hyperproliferation, slow tumor growth and/or reduce tumor volume.

In certain embodiments of the method, the at least one metal cation is contained in at least one metal complex represented by formula (I):

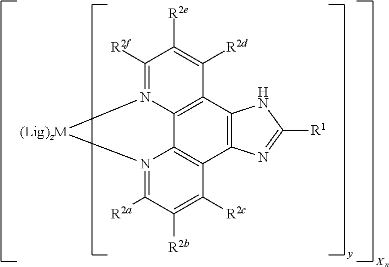

including hydrates, solvates, pharmaceutically acceptable salts and prodrugs thereof, wherein:
the at least one metal cation (M) is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
y=1,2, or 3;
z=0, 1, or 2;
Lig at each occurrence is independently selected from the group consisting of

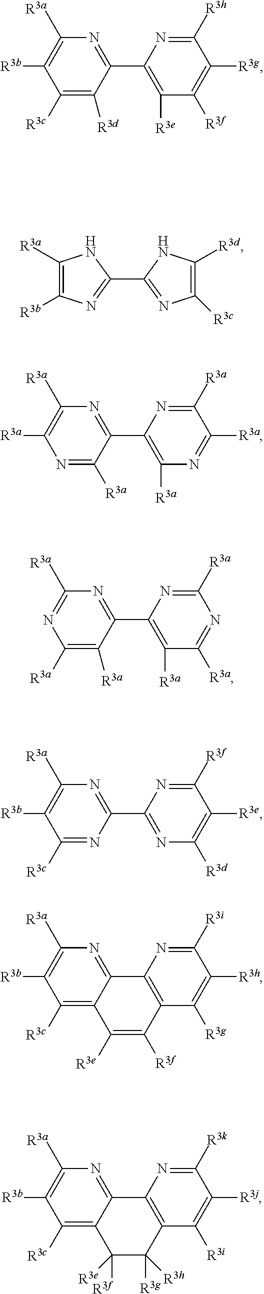

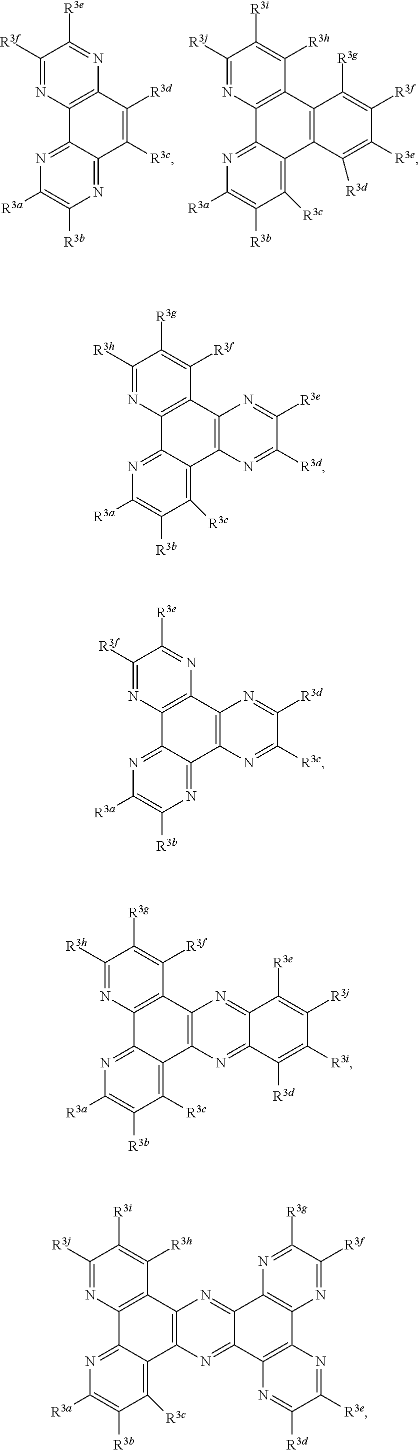

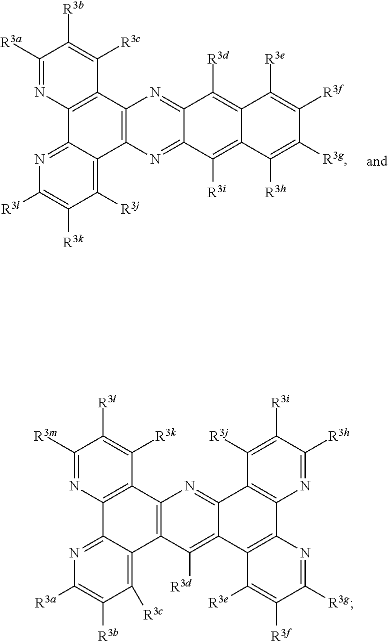

R¹ is selected from the group consisting of

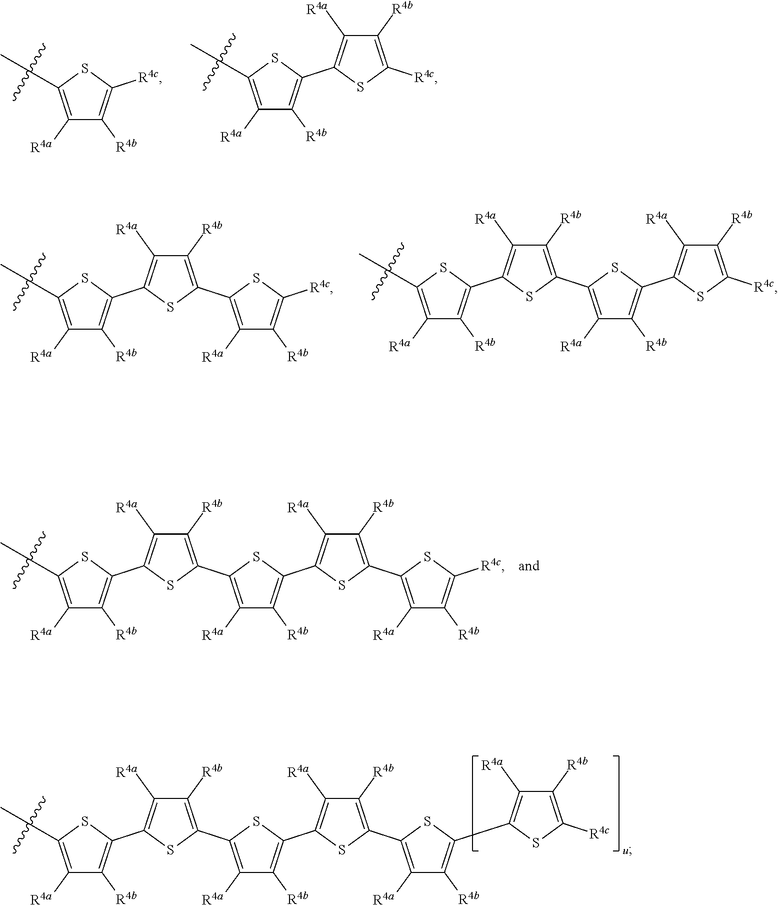

u is an integer of 1 to 20;
R^{2a}, R^{2b}, R^{2c}, R^{2d}, R^{2e}, and R^{2f} at each occurrence are each independently selected from the group consisting of hydrogen, C1-6 optionally substituted alkyl, C1-6 optionally substituted branched alkyl, C3-7 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h}, R³ⁱ, R^{3j}, R^{3k}, R^{3l}, and R^{3m} at each occurrence are each independently selected from the group consisting of hydrogen, C1-6 optionally substituted alkyl, C1-6 optionally substituted branched alkyl, C1-6 optionally substituted haloalkyl, C1-6 optionally substituted alkoxy, and CO₂R⁸;
R^{4a}, R^{4b}, and R^{4c} at each occurrence are each independently selected from the group consisting of hydrogen, C1-6 optionally substituted alkyl, C1-6 optionally substituted branched alkyl, C1-6 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;
R⁶ at each occurrence is independently selected from the group consisting of hydrogen and optionally substituted alkyl;
R⁷ at each occurrence is independently selected from the group consisting of hydrogen and optionally substituted alkyl; and
R⁸ at each occurrence is independently selected from the group consisting of hydrogen and optionally substituted alkyl.

In certain embodiments of the method, the composition is formed in vivo by administering the at least one metal complex and the at least one infectious agent separately.

In certain embodiments of the method, the composition is formed prior to being administered to the patient.

In certain embodiments of the method, the condition is non-muscle invasive bladder cancer.

In certain embodiments, the method further comprises administering transferrin to the patient.

In certain embodiments of the method, the at least one metal complex comprises at least one member selected from the group consisting of:
Ru(2,2′-bipyridine)₂(2-(2′,2″:5″,2′″-terthiophene)-imidazo[4,5-][1,10]phenanthroline);
Ru(2,2′-bipyridine)₂(2-(2′,2″:5″,2′″;5′″,2″″-quaterthiophene)-imidazo[4,5-f][1,10]phenanthroline);
Ru(4,4′-dimethyl-2,2′-bipyridine)₂(2-thiophenimidazo[4,5-f][1,10]phenanthroline);
Ru(4,4′-dimethyl-2,2′-bipyridine)₂(2-(2′,2″-bithiophene)-imidazo[4,5-f][1,10]phenanthroline);
Ru(4,4′-dimethyl-2,2′-bipyridine)₂(2-(2′,2″:5″,2′″-terthiophene)-imidazo[4,5-f][1,10]phenanthroline);
Ru(4,4′-dimethyl-2,2′-bipyridine)₂(2-(2′,2″:5″,2′″;5′″,2″″-quaterthiophene)-imidazo[4,5-f][1,10]phenanthroline);
Ru(4,4′-di-t-butyl-2,2′-bipyridine)₂(2-thiophenimidazo[4,5-f][1,10]phenanthroline);
Ru(4,4′-di-t-butyl-2,2′-bipyridine)₂(2-(2′,2″-bithiophene)-imidazo[4,5-f][1,10]phenanthroline);
Ru(4,4′-di-t-butyl-2,2′-bipyridine)₂(2-(2′,2″:5″,2′″-terthiophene)-imidazo[4,5-f][1,10]phenanthroline);
Ru(4,4′-dimethoxy-2,2′-bipyridine)₂(2-(2′,2″:5″,2′″-terthiophene)-imidazo[4,5-f][1,10]phenanthroline);
Ru(5,5′-dimethyl-2,2′-bipyridine)₂(2-thiophenimidazo[4,5-f][1,10]phenanthroline);
Ru(5,5′-dimethyl-2,2′-bipyridine)₂(2-(2′,2″-bithiophene)-imidazo[4,5-f][1,10]phenanthroline);
Ru(5,5′-dimethyl-2,2′-bipyridine)₂(2-(2′,2″:5″,2′″-terthiophene)-imidazo[4,5-f][1,10]phenanthroline);
Ru(6,6′-dimethyl-2,2′-bipyridine)₂(2-thiophenimidazo[4,5-f][1,10]phenanthroline);
Ru(6,6′-dimethyl-2,2′-bipyridine)₂(2-(2′,2″-bithiophene)-imidazo[4,5-f][1,10]phenanthroline);
Ru(6,6′-dimethyl-2,2′-bipyridine)₂(2-(2′,2″:5″,2′″-terthiophene)-imidazo[4,5-f][1,10]phenanthroline);
Ru(4,4′-di(methylcarboxy)-2,2′-bipyridine)₂(2-(2′,2′″:5″,2′″-terthiophene)-imidazo[4,5-f][1,10]phenanthroline);
Ru(2,2′-bipyrimidine)₂(2-(2′,2″:5″,2′″-terthiophene)-imidazo[4,5-f][1,10]phenanthroline);
Ru(2,2′-bipyrimidine)(4,4′-dimethyl-2,2′-bipyridine)(2-(2′,2′″:5″,2′″-terthiophene)-imidazo[4,5-f][1,10]phenanthroline);
Ru(1,10-phenanthroline)₂(2-thiophenimidazo[4,5-f][1,10]phenanthroline);
Os(2,2′-bipyridine)₂(2-thiophenimidazo[4,5-f][1,10]phenanthroline);
Os(2,2′-bipyridine)₂(2-(2′,2″-bithiophene)-imidazo[4,5-f][1,10]phenanthroline);
Os(2,2′-bipyridine)₂(2-(2′,2″:5″,2′″-terthiophene)-imidazo[4,5-f][1,10]phenanthroline);
Os(1,10-phenanthroline)₂(2-thiophenimidazo[4,5-f][1,10]phenanthroline);
Os(1,10-phenanthroline)₂(2-(2′,2″-bithiophene)-imidazo[4,5-][1,10]phenanthroline);
Os(1,10-phenanthroline)₂(2-(2′,2″:5″,2′″-terthiophene)-imidazo[4,5-][1,10]phenanthroline);
Os(4,4′-dimethyl-2,2′-bipyridine)₂(2-thiophenimidazo[4,5-f][1,10]phenanthroline);
Os(4,4′-dimethyl-2,2′-bipyridine)₂(2-(2′,2″-bithiophene)-imidazo[4,5-f][1,10]phenanthroline);
Os(4,4′-dimethyl-2,2′-bipyridine)₂(2-(2′,2′″:5″,2′″-terthiophene)-imidazo[4,5-f][1,10]phenanthroline); and
pharmaceutically acceptable salts thereof.

In certain embodiments of the method, the at least one metal complex is Ru(4,4′-dimethyl-2,2′-bipyridine)₂(2-(2′,2″:5″,2′″-terthiophene)-imidazo[4,5-f][1,10]phenanthroline) or a pharmaceutically acceptable salt thereof.

In certain embodiments of the method, a single dose of the composition contains 0.001 μg to 80000 μg of the at least one metal cation and 1×10⁴ to 8×10⁻⁸ CFU of the at least one infectious agent.

In certain embodiments of the method, the at least one infectious agent comprises engineered bacteria selected from the group consisting of live attenuated Mycobacterium Bovis, engineered Salmonella, engineered Staphylococcus, engineered Listeria, engineered E-coli, engineered Bifidobacterium, and engineered Clostridium.

In certain embodiments, the method further comprises administering at least one negatively charged substance additional to the at least one infectious agent, wherein the negatively charged substance is at least one of a lipopolysaccharide and a negatively charged microorganism.

In certain embodiments of the method, the administering step comprises administering to the patient at least two different infectious agents selected from the group consisting of bacteria, protozoa, fungi, viruses, algae and structural components thereof.

Other features and advantages of the present invention will become apparent from the following detailed description, examples and figures. It should be understood; however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF SEVERAL VIEWS OF THE DRAWINGS

The invention will be described in conjunction with the following drawings, wherein:

FIG. 1 is a bar graph showing the toxicity effect of BCG alone in T24 cells.

FIG. 2A shows spectral measurements of the optical density against wavelength of BCG alone, RUVIDAR alone and BCG plus RUVIDAR.

FIG. 2B is a spectrum showing the binding signature of RuBCG produced from 3.2 mg/mL BCG mixed with 1 mM RUVIDAR in water.

FIG. 3 is a bar graph showing the zeta potential of RuBCG prepared in water from 0.04 mg/mL BCG and 2 or 20 μM RUVIDAR.

FIG. 4 is a bar graph showing the time course stability of zeta potential of RuBCG prepared in water from 0.04 mg/mL BCG and 20 μM RUVIDAR.

FIG. 5 is a bar graph showing the synergistic toxicity of RuBCG in T24 cells relative to a control, BCG alone and RUVIDAR alone.

FIG. 6A is a graph of human colon adenocarcinoma tumor volume over time, which shows the in vivo efficacy of RuBCG relative to a control, BCG alone and RUVIDAR alone.

FIG. 6B is a bar graph showing percent survival of human colon adenocarcinoma tumors, which were untreated, or treated with RuBCG, BCG alone or RUVIDAR alone.

FIG. 7 is a bar graph showing the effects of a control, RUVIDAR alone, BCG alone and RuBCG on the percent control of PDL-1 signal.

FIG. 8A is a graph of human colon adenocarcinoma tumor volume over time, which shows the in vivo efficacy of RUTHERRIN-based RuBCG relative to a control, BCG alone and RUTHERRIN alone.

FIG. 8B is a bar graph showing percent survival of human colon adenocarcinoma tumors, which were untreated, or treated with RUTHERRIN-based RuBCG, BCG alone or RUTHERRIN alone.

FIG. 9 is a bar graph showing binding of Fe³⁺ to BCG cells manifested by the inversion of zeta potential, wherein the data are presented as Mean±SEM, N=4-6.

FIG. 10A is a spectrum of Optical Density against wavelength of BCG alone.

FIG. 10 B shows spectra of Fe³⁺ alone and FeBCG.

FIG. 10C is a spectrum showing the binding signature of FeBCG.

FIG. 11 is a bar graph showing the cytotoxicity of FeBCG in T24 cells with the toxicity of BCG alone and Fe³⁺ alone shown as references.

### DETAILED DESCRIPTION OF PREFERRED EMBODIMENTS OF THE INVENTION

### Glossary

Throughout the description, where compositions are described as: having, including or comprising specific components or where processes are described as: having, including or comprising specific process steps, it is contemplated that compositions of the present teachings also consist essentially of, or consist of, the recited components and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial, so long as the present teachings remain operable. Moreover, two or more steps or actions can be conducted simultaneously.

For the purposes of the present invention the terms “compound”, “complex”, “metal complex” and “composition of matter” stand equally well for the inventive complexes described herein, be they photodynamic or not, including all enantiomeric forms, diastereomeric forms, salts and the like and the terms “compound”, “complex”, “metal complex” and “composition of matter” are used interchangeably throughout this specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to: diastereomeric salt formation, kinetic resolution and asymmetric synthesis. The present teachings also encompass cis and trans isomers of compounds containing alkenyl moieties (e.g.: alkenes and imines). It is also understood that the present teachings encompass all possible regioisomers, and mixtures thereof, which can be obtained in pure form by standard separation procedures known to those skilled in the art, and include, but are not limited to: column chromatography, thin-layer chromatography and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include: metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g.: ethyl-tert-butyl-, diethyl-, diisopropyl-, triethyl-, tributyl- or dimethylpropylamine), or a mono-, di-, or trihydroxy lower alkylamine (e.g.: mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic, as well as other known pharmaceutically acceptable acids.

When any variable occurs more than one time in any constituent or in any formula, its definition in each occurrence is independent of its definition at every other occurrence (e.g.: in N(R⁶)₂, each R⁶ may be the same or different than the other). Combinations of substituents and/or variables are permissible only if such combinations result in stable compounds.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

As used herein, the term “photodynamic therapy” or “PDT” refers to a treatment for destroying cells or modulating immune function, including immune response, of cells and tissue through use of a drug that can be activated by light of a certain wavelength and dose.

As used herein, the term “radiodynamic therapy” or “RDT” refers to a treatment for destroying cells or modulating immune function, including immune response, of cells and tissue through use of a drug that can be activated by ionizing radiation of a certain wavelength and dose.

As used herein, the term “photodynamic compound” or “PDC” refers to a compound that can be activated by light of a certain wavelength and dose for PDT. The term is also used herein to refer to a compound that can be activated by ionizing radiation of a certain wavelength and dose for RDT.

As used herein, the term “radiation” used without the term “ionizing” is intended to encompass all types of radiation in the electromagnetic spectrum including light and ionizing radiation.

As used herein, the term “immunotherapy” refers to a treatment which elicits an immune response from a patient so as to prevent, ameliorate or cure a condition (e.g., a disease or an infection).

An “immunotherapeutic agent” is a substance that elicits an immune response from a patient, so as to prevent, ameliorate or cure a condition (e.g.: a disease or an infection).

Except when noted, the terms “subject” or “patient” are used interchangeably and refer to mammals such as human patients and non-human primates, as well as experimental animals such as: dogs, rabbits, rats, mice and other animals. Accordingly, the term “subject” or “patient” as used herein means any mammalian patient or subject to which the compounds of the invention can be administered. In an exemplary embodiment of the present invention, to identify subjects or patients for treatment according to the methods of the invention, accepted screening methods are employed to determine risk factors associated with a targeted or suspected disease or condition or to determine the status of an existing disease or condition in a subject or patient. These screening methods include, for example: conventional work-ups to determine risk factors that may be associated with the targeted or suspected disease or condition. These and other routine methods allow the clinician to select patients in need of therapy using the methods and compounds of the present invention.

As used herein, the expression “biological target” refers to an organ, tissue and/or cell of an organism and/or to the organism itself.

As used herein the term “immunogenic” refers to a substance that is able to elicit an immune response.

The term “substituted” means that an atom or group of atoms formally replaces hydrogen as a “substituent” attached to another group. The term “substituted”, unless otherwise indicated, refers to any level of substitution, e.g.: mono-, di-, tri-, tetra- or penta-substitution, where such substitution is permitted. The substituents are independently selected and substitution may be at any chemically accessible position. It is to be understood that substitution at a given atom is limited by valency. It is to be understood that substitution at a given atom results in a chemically stable molecule. The phrase “optionally substituted” means unsubstituted or substituted. A single divalent substituent, e.g.: oxo, can replace two hydrogen atoms.

The term “alkyl” employed alone or in combination with other terms, refers to a saturated hydrocarbon group that may be straight-chained or branched. Examples of alkyl moieties include, but are not limited to: chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. Examples of alkenyl groups include, but are not limited to: ethenyl, n-propenyl, isopropenyl, n-butenyl, sec-butenyl and the like.

The term “alkoxy”, employed alone or in combination with other terms, refers to a group of formula —O-alkyl, wherein the alkyl group is as defined above. Examples of alkoxy groups include: methoxy, ethoxy, propoxy (e.g.: n-propoxy and isopropoxy), t-butoxy and the like.

All compounds, and pharmaceutically acceptable salts thereof, can be found together with other substances such as water and solvents (e.g.: hydrates and solvates) or can be isolated. When in the solid state, the compounds described herein and salts thereof may occur in various forms and may, for example, take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive: toxicity, irritation, allergic response, or other problem or complication, commensurate with a reasonable benefit/risk ratio.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to: mineral or organic acid salts of basic residues such as amines; alkali or organic salts of acidic residues such as carboxylic acids; and the like. The pharmaceutically acceptable salts of the present invention include the non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like: ether, ethyl acetate, alcohols (e.g.: methanol, ethanol, iso-propanol or butanol) or acetonitrile (MeCN) are preferred. Lists of suitable salts are found in Remington's Pharmaceutical Sciences, 17th Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection and Use, (Wiley, 2002). In some embodiments, the compounds described herein include the N-oxide forms.

The term “infectious agent” as used herein refers to organisms capable of infecting another organism. Infectious agents can be microscopic or visible to the naked eye. The term encompasses pathogens, but as used herein, infectious agents need not have a deleterious effect on the health of the host organism. Non-limiting examples of infectious agents include bacteria, protozoa, fungi, viruses, algae and structural components thereof. The term “structural components” as used herein refers to organized structures at the cellular level, such as, e.g., cell membranes, cytoplasm, nucleus, organelles and cell walls, as opposed to molecular structures, such as, e.g., proteins, lipids, carbohydrates, nucleic acids and the like.

All percentages, ratios and proportions herein are by weight, unless otherwise specified. All temperatures are in degrees Celsius (° C.) unless otherwise specified.

The present disclosure provides, among other things, compositions and methods for treating conditions associated with cell hyperproliferation, such as, e.g., cancerous disorders including but not limited to bladder cancer, e.g., low-grade, high-grade, BCG-unresponsive non-muscle-invasive bladder cancer (NMIBC)). Certain embodiments of the inventive compositions comprise: (i) a positively charged metal-based compound (e.g., a transition-metal based coordination complex, which is mimicking iron properties to binding with transferrin, as well as ferroptosis alike redox/oxidative potential, and (ii) engineered bacteria effective to treat cancer. The compositions can be used alone or in combination with other therapeutic agents, procedures, or modalities to treat or prevent cell hyperproliferation disorders, including but not limited to cancer.

### Therapeutic Method

The method of the invention preferably comprises: (a) administering to a patient having a condition associated with hyperproliferation of cells at least one infectious agent selected from the group consisting of bacteria, protozoa, fungi, viruses and algae, wherein the at least one infectious agent has a net negative charge and is effective to treat the condition; and (b) administering to the patient before, during and/or after step (a) at least one metal cation, wherein the method is effective to slow cell hyperproliferation, slow tumor growth and/or reduce tumor volume. In certain embodiments, the method of the invention preferably comprises administering to a patient a composition to treat cancer in the patient, wherein the composition comprises engineered bacteria and a metal complex represented by Formula (I) as defined above, including hydrates, solvates, pharmaceutically acceptable salts and prodrugs thereof.

Without wishing to be bound by any theory, it is believed that the inventive composition at least partially reverses or at least partially prevents resistance to an original or primary treatment that is compromised or is susceptible to being compromised by, e.g., MDR, by blocking the specific mechanism of resistance so as to “re-sensitize” the patient to the original treatment. In some embodiments, the composition is useful for the inhibition of efflux pump activity and re-sensitizing targeted cells (e.g., infected cells and/or cancer cells) to drugs and/or other forms of active therapy.

It is further believed that preferred embodiments of the invention induce “Rutherroptosis”, which as defined herein is a process that mimics iron-induced ferroptosis, using metal cation substances, such as, e.g., complexes according to Formula (I), wherein the metal is ruthenium and the ligands include phenanthroline and thiophene. Particularly preferred among these complexes is Ru(4,4′-dimethyl-2,2′-bipyridine)₂(2-(2′,2″:5″,2′″-terthiophene)-imidazo[4,5-f][1,10]phenanthroline), which is available from Theralase Technologies of Toronto, Canada, as a dichloride salt with (RUTHERRIN) or without transferrin (RUVIDAR).

Inert metal polypyridyl complexes of Formula (I) with bacteria hold tremendous potential as chemotherapeutic agents against cancer, both in the presence of light and/or radiation—i.e., as sensitizers for PDT and RDT—and in their absence.

Hence, in certain embodiments, the present invention provides a method of treating a drug resistant tumor and/or recurrent tumors comprising administering a metal complex of Formula (I) to a mammalian patient suffering from these disorders under conditions effective to treat the drug resistant tumor and/or persistent tumors via induction of a particular intracellular process, which is (to at least some extent) mimicking the ferroptosis pathway.

In normal cells, reactive oxygen species (ROS) levels are kept low due to the antioxidant systems that maintain redox balance due to oxidative phosphorylation metabolic pathway (Schumacker, 2015). In cancer cells, due to aerobic glycolysis, Warburg effect, the level of ROS increases to meet the need of malignant proliferation and progression, but may still stay below the threshold to avoid cytotoxicity (Marengo et al., 2016).

RUVIDAR and RUTHERRIN have been shown to exhibit a lower systemic toxicity and are linked with a higher selectivity towards, particularly stem and/or high grade, hypoxic cancer cells, which is knowingly expressing the highest level of transferrin receptor. See Gao et al., 2023.

Without activation by light and/or radiation, the activation for these highly versatile metal-ligands complexes in cancer cells can be driven by high redox/oxidative potential, which could carry out their significant cytotoxic activity in cancer cells through interactions with the cellular redox/oxidative Warburg's homeostasis.

Therefore, in yet another aspect, the present invention provides a method of treating a drug resistant tumor and/or recurrent tumors comprising administering: (a) metal cations and/or a complex of Formula (I) containing a metal cation; and (b) at least one infectious agent (e.g., engineered bacteria) to a patient suffering from these disorders under conditions effective to treat the drug resistant tumor and/or persistent tumors via induction of redox/oxidative intracellular process under hypoxia and/or anaerobic conditions.

Radiotherapy uses radiation to irradiate tumor tissues and kill tumor cells. On the one hand, radiation acts directly on cells and instantly produces a large number of free radicals. On the other hand, it indirectly produces lasting and severe therapeutic effects through the redox reaction of water (Spitz et al., 2004).

Due to the high content of water in cells, when water absorbs the energy of low-LET rays, a redox reaction occurs, and a large number of free radicals and free electrons are produced. The free radicals and electrons generated initiate cascade reactions that produce OH, H₂O₂, and O_{2′}⁻, significantly increasing the level of ROS (Bielski et al., 2009). Notably, oxidative changes can persist for several months after initial radiotherapy. This feature is related to the continuous generation of ROS and its heritability in the offspring of irradiated cells and obviously enhances the curative effect (Tamminga et al., 2011).

The basic idea behind activation of metal complexes, such as RUVIDAR and RUTHERRIN, is based on the induction of photoconversion of the metal complexes that triggers the production of ROS and free radicals, leading to the killing of unwanted cells. The main difficulties related to use of light-based PDT for clinical applications are due to the intrinsic process, such as absorption and scattering of light in penetrating tissues, the low concentration of FDA-approved PDCs, particularly pro-drugs like ALA, which must be effectively converted to (under the Warburg's metabolic environment with the lack of oxygen) to the active pharmaceutical ingredient PPIX.

Upon the higher charge particle energy about >633 KeV emitted by different radiation sources the Vavilov-Cherenkov effect (photon emission), which is possible to enhance the radiation effect on activation of PDCs, such as RUVIDAR and RUTHERRIN, is observed. A classic example of Cherenkov radiation is the characteristic blue glow of an underwater nuclear reactor. Its cause is similar to the cause of a sonic boom, the sharp sound heard when faster-than-sound and/or light movement occurs (Cherenkov, 1934).

The mainstream treatment for cancer, chemotherapy, also often works by changing the redox state of cancer cells. Quite a few chemotherapeutics induce oxidative stress and ROS-mediated cell damage in cancer cells by increasing ROS above the threshold to yield an anticancer effect (Pelicano et al., 2004). Most of these drugs produce ROS directly in cancer cells to increase the level of ROS. The first drug developed to achieve therapeutic effects by producing ROS was procarbazine. Procarbazine can be oxidized in aqueous solution and produce H₂O₂ and .OH.

When coordinating with ionizing radiation, procarbazine forms unstable peroxides to damage DNA in vitro (Berneis et al., 2004). It was approved for the treatment of primary brain tumours and other diseases 60 years ago (Behrend, 2014).

Nowadays, drugs like anthracycline (Songbo et al., 2019) are widely used in cancer treatment to promote ROS production.

Chemically, oxidative stress is the result of an imbalance between the production and consumption of oxidants. Bladder cancer cells internalize BCG, which will induce inducible nitric oxide synthase (iNOS) to produce nitric oxide (NO) (Galano et al., 2018). The cytotoxic effect of the high dose of NO on urothelial carcinoma cells has been reported for a long time (Ryk et al., 2015, Severino et al. 2018, Thiel et al. 2014). Besides, the active BCG can stimulate the production of ROS such as hydrogen peroxide (H₂O₂), which can further produce NO (Shah et al., 2014) The production of these substances will interact with each other to produce strong oxidants, leading to oxidative stress response in tumor cells. In turn, the oxidative stress response can cause more production of ROS and NOS. This reaction eventually leads to the damage of DNA and proteins in cells or causes a series of effects such as cell apoptosis and autophagy (Shah, Zhang et al. 2014).

Therefore, in yet another aspect, the present invention provides a method of treating a drug resistant tumor and/or recurrent tumors comprising administering: (a) metal cations and/or a compound of Formula (I) containing a metal cation; and (b) at least one infectious agent (e.g., engineered bacteria) to a mammalian patient suffering from these disorders under conditions effective to treat the drug resistant tumor and/or persistent tumors via induction of redox/oxidative intracellular process under hypoxia and/or anaerobic conditions. In some embodiments, said administering is carried out in combination with another cancer therapy.

Tumor cells develop various mechanisms to resist immune attack. The immune pressure exerted against the tumor during immunotherapy treatments is strongly suggested by the escape mechanisms developed by the tumor, especially against the attack of cytotoxic T lymphocytes (De Guillebon et al., 2020).

This immune resistance can be reversed and/or uniquely modulated by the dual checkpoint inhibition (e.g., CD47 and PD-Li inhibition) with administering a compound of formula (I) to a mammalian patient. This inventive therapeutic approach will also improve patient outcomes over current PD-L1 and CD47-targeted therapies.

Hence, in some embodiments, the method and composition of the invention are useful for the inhibition of several immunologic checkpoints and re-sensitizing the cancer cells and/or cancer-resistant cells to drugs and/or other forms of active therapy and/or immunotherapy. In preferred embodiments, said administering is carried out in combination with another cancer or anti-infective therapy (e.g., at least one chemotherapeutic agent and/or light and/or other radiation).

Certain embodiments of the method comprise treating a low-grade, high-grade, BCG-unresponsive non-muscle-invasive bladder cancer (NMIBC) in a subject, comprising administering a composition of the invention to the subject intravesically once a week for 6 weeks, at least for a year, started a few weeks after a transurethral resection of bladder tumor (TURBT).

In certain embodiments, the method further comprises administering to the subject at least one additional therapeutic agent. In certain embodiments, the at least one additional therapeutic agent comprises a checkpoint inhibitor. In certain embodiments, the checkpoint inhibitor targets a checkpoint molecule chosen from PD-1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD 160, 2B4, CD80, CD86, B7-H3 (CD276), B7-H4 (VTCN1), HVEM (e.g., TNFRSF14 or CD270), KIR, A2aR, MHC class I, MHC class II, GAL9, adenosine, or TGF (e.g., TGF beta). In certain embodiments, the checkpoint inhibitor comprises or is an anti-PD-1 antibody or a fragment thereof, e.g., Pembrolizumab. In certain embodiments, Pembrolizumab is administered intravenously about every three weeks or longer than every three weeks (e.g., every four weeks, every five weeks, every six weeks, or more).

In certain embodiment, the method further comprises administrating to the subjects at least one additional therapeutic agent selected from a non-replicating recombinant viral gene therapy or an attenuated recombinant gene therapy and/or gene therapy.

Negatively charged, purified lipopolysaccharide (LPS) was confirmed to have positive antitumor activity in humans; however, both the toxicity of LPS as well as the relatively rapid induction of tolerance by LPS detracted from its overall utility as a cancer chemotherapeutic (Rockwell et al., 2010). Moreover, metal-based, phenanthroline, thiophene compounds like RUVIDAR and RUTHERRIN can be conjugated or premixed with other attenuated, negatively charged microorganisms, for example, Clostridium novyi, etc.

Hence, another embodiment of the inventive method comprises treating, suppressing, reducing the severity, reducing the risk of treatment failure, or inhibiting cancer comprising administering: (a) metal cations and/or a complex of Formula (I) containing a metal cation; and (b) at least one infectious agent (e.g., engineered bacteria) as a premix or conjugate with LPS to a mammalian patient having cancer under conditions effective to treat the cancer. In certain embodiments, the metal complex is conjugated or premixed with an attenuated, negatively-charged strain of various microorganisms, such as Clostridium novyi. In certain embodiments, the method is carried out in combination with another cancer therapy.

### Composition Comprising the Metal Cations and Infectious Agent

The composition for treating a condition associated with hyperproliferation of cells preferably comprises at least one infectious agent having a net negative charge and effective to treat the condition; and at least one metal cation in an amount effective to more than offset the net negative charge of the at least one infectious agent such that the composition has a net positive charge.

The at least one metal cation is a positively charged ionic species of a metal preferably selected from the group consisting of manganese, molybdenum, rhenium, iron, ruthenium, osmium, cobalt, rhodium, iridium, nickel, platinum, and copper. The at least one metal cation is preferably contained in at least one metal complex represented by formula (I) as defined above.

The at least one infectious agent is preferably selected from the group consisting of bacteria, protozoa, fungi, viruses, algae and structural components thereof. In certain embodiments, the at least one infectious agent is naturally occurring and in other embodiments the at least one infectious agent is not naturally occurring (or “engineered”). In certain embodiments, the engineered infectious agent is attenuated relative to its naturally occurring form. For example, BCG comprises live attenuated Mycobacterium bovis as the engineered bacteria. Engineered infectious agents also encompass genetically modified infectious agents.

Bacteria are preferably selected from the group consisting of live attenuated Mycobacterium Bovis, engineered Salmonella, engineered Staphylococcus, engineered Listeria, engineered E-coli, engineered Bifidobacterium, and engineered Clostridium.

Protozoa are preferably selected from the group consisting of Leishmania infantum and Leishmania tropica, Neospora caninum and Toxoplasma gondii.

Fungi are preferably selected from the group consisting of Lachnum, Agaricus blazei Murill and Cordyceps militaris.

Non-limiting examples of viruses suitable for use in the invention include Adenoviruses, Adeno-associated virus, Herpes viruses, Pox viruses, Lentiviruses, Retroviruses, Alphaviruses, Newcastle disease virus, Picornavirus and Measles virus. See, e.g., Umair et al. (2022). Viruses as tools in gene therapy, vaccine development, and cancer treatment. Archives of Virology, 167(6), 1387-1404 for additional non-limiting examples thereof.

Algae are preferably selected from the group consisting of Chlorella vulgaris, brown algae and blue-green micro algae.

It is believed that several properties make bacteria suitable for cancer treatment, including their unique form of motility, which allows them to easily penetrate tumors; their ability to thrive in the hypoxic and immune-deficient environments of tumors; and their ability to deplete the tumor microenvironment of nutrients necessary for cancer cell survival. Moreover, microorganisms, such as bacteria, can modulate anticancer inflammatory responses and activate anti-cancer immunity.

Negatively charged infectious agents (e.g., bacteria) can ionically bond to positively charged metal and complexes containing said metal cation. In certain embodiments, the at least one infectious agent can covalently bond to a metal (e.g., Iron (Fe³⁺) or metal complex.

For the purposes of the present invention the terms “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable and functional pharmaceutical, serving not only as part of the overall vehicle for delivery, but also as a means for achieving effective absorption by the recipient of the active ingredient. An excipient may fill a role as simple and direct as being an inert filler, or an excipient as used herein may, for example, be part of a pH stabilizing system or coating to insure delivery of the ingredients safely to the tissue.

Examples of such excipients or carriers are well known to those skilled in the art and can be prepared in accordance with acceptable pharmaceutical procedures, such as, for example, those described in Remington's Pharmaceutical Sciences, 17^{th} edition, ed. Alfonoso R. Gennaro, Mack Publishing Company, Easton, PA (1985), the entire disclosure of which is incorporated by reference herein for all purposes. Supplementary active ingredients can also be incorporated into the pharmaceutical compositions.

The composition of the invention can be administered: orally, intravenously, intravesically, lingually, intratumorally, topically or parenterally, neat or in combination with conventional pharmaceutical carriers. Applicable solid carriers can include one or more substances which can also act as: flavoring agents, lubricants, solubilizers, suspending agents, fillers, glidants, compression aids, binders or tablet-disintegrating agents or encapsulating materials. The active agents (e.g., engineered bacteria and the metal complex of Formula I) can be formulated in a conventional manner, for example, in a manner similar to that used for known active agents. Oral formulations containing an active agent disclosed herein can comprise any conventionally used oral form, including: tablets, capsules, buccal forms, troches, lozenges and oral liquids, suspensions or solutions. In powders, the carrier can be a finely divided solid, which is an admixture with a finely divided active agent. In tablets, an active agent disclosed herein can be mixed with a carrier having the necessary compression properties in suitable proportions and compacted in the shape and size desired. The powders and tablets can contain up to 99% of the active agent.

Capsules can contain mixtures of one or more compound(s) and/or compositions disclosed herein with inert filler(s) and/or diluent(s) such as: pharmaceutically acceptable starches (i.e.: corn, potato or tapioca starch), sugars, artificial sweetening agents, powdered celluloses (i.e.: crystalline and microcrystalline celluloses), flours, gelatins, gums and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to: magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes and ion exchange resins. Surface modifying agents include nonionic and anionic surface modifying agents. Representative examples of surface modifying agents include, but are not limited to: poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate and triethanolamine. Oral formulations herein can utilize standard delay or time-release formulations to alter the absorption of the compound(s) and/or compositions. The oral formulation can also consist of administering an active agent disclosed herein in water or fruit juice, containing appropriate solubilizers or emulsifiers, as needed.

Liquid carriers can be used in preparing solutions, suspensions, emulsions, syrups, elixirs and for inhaled delivery. An active agent of the invention can be dissolved or suspended in a pharmaceutically acceptable liquid carrier such as: water, an organic solvent, a mixture of both or a pharmaceutically acceptable oil or fat. The liquid carrier can contain other suitable pharmaceutical additives such as: solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to: water (particularly containing additives as described herein, for example, cellulose derivatives such as a sodium carboxymethyl cellulose solution), alcohols (including monohydric alcohols and polyhydric alcohols, for example, glycols) and their derivatives, and oils (i.e.: fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example: intramuscular, intraperitoneal, topical or subcutaneous injection. Sterile solutions can also be administered intravenously. Compositions for oral administration can be in either liquid or solid form.

In certain embodiments, the pharmaceutical composition is in unit dosage form, for example as: tablets, capsules, powders, solutions, suspensions, emulsions, granules or suppositories. In such form, the pharmaceutical composition can be sub-divided into unit dose(s) containing appropriate quantities of the active agent. The unit dosage forms can be packaged compositions, for example: packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 0.001 μg to 80000 μg of the at least one metal complex and 1×10⁻⁴ to 8×10⁻⁸ CFU of the at least one infectious agent, and can be given in a single dose or in two or more doses. Such doses can be administered in any manner useful in directing the compound(s) and/or composition(s) to the target tissue and/or bloodstream of the patient, including: orally, via implants, parenterally (including intravenous, intraperitoneal, topical and subcutaneous injections), rectally, vaginally and transdermally.

In certain embodiments it is envisioned that the dosage of at least one of the metal and/or (properly) inoculated organic composition may vary from between about 0.001 g compound/kg body weight to about 80 mg/kg body weight.

In other embodiments the intravesical doses of at least one of the metal and/or organic composition may be about 50, 100, 200, 300, 400, 500, 600, 700, 800, 900, 1000, 2000, 3000, 4000, 5000, 6000, 7000, 8000, 9000 or 10000 to 20000, 40000, 60000 or 80000 μg/mL.

Of course, this dosage amount may be adjusted upward or downward, as is routinely done in such treatment protocols, depending on the results of the initial clinical trials and the needs of a particular patient.

When administered for the treatment or inhibition of a particular disease state or disorder, it is understood that an effective dosage can vary depending upon the particular active agent utilized, the mode of administration and severity of the condition being treated, as well as the various physical factors related to the individual being treated. In therapeutic applications, an active agent can be provided to a patient already suffering from a disease in an amount sufficient to heal or at least partially ameliorate the symptoms of the disease and its complications. The dosage to be used in the treatment of a specific patient typically must be subjectively determined by the attending physician. The variables involved include the specific condition and its state as well as the: physical size, age, gender, health status and response pattern of the patient.

In some cases, it may be desirable to administer the active agents directly to the airways of the patient, using devices such as, but not limited to: metered dose inhalers, breath-operated inhalers, multidose dry-powder inhalers, pumps, squeeze-actuated nebulized spray dispensers, aerosol dispensers and aerosol nebulizers. For administration by intranasal or intrabronchial inhalation, the active agent(s) can be formulated into a liquid composition, a solid composition, or an aerosol composition. The liquid composition can include, by way of illustration, one or more active agents dissolved, partially dissolved or suspended in one or more pharmaceutically acceptable solvents and can be administered by, for example, a pump or a squeeze-actuated nebulized spray dispenser. The solvents can be administered by, for example, isotonic saline or bacteriostatic water. The solid composition can be administered, by way of illustration, a powder preparation including one or more active agents intermixed with lactose or other inert powders that are acceptable for intrabronchial use, and can be administered by, for example, an aerosol dispenser or a device that breaks or punctures a capsule encasing the solid active agent and delivers the solid active agent for inhalation. The aerosol active agent can include, by way of illustration, one or more active agents, propellants, surfactants and co-solvents, and can be administered by, for example, a metered device. The propellants can be a chlorofluorocarbon, a hydrofluoroalkane, or other propellants that are physiologically and environmentally acceptable.

The active agents of the invention can be administered parenterally or intraperitoneally. Solutions or suspensions of these active agents or pharmaceutically acceptable salts, hydrates, or esters thereof can be prepared in water suitably mixed with a surfactant such as hydroxylpropylcellulose. Dispersions can also be prepared in propylene glycol, glycerol, liquid polyethylene glycols and/or mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In certain embodiments, the form can be sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example: water, ethanol, polyol (i.e.: propylene glycol, glycerol and liquid polyethylene glycol) and/or suitable mixtures thereof in oils.

Active agents described herein can be administered transdermally (i.e.: administered across the surface of the body and the inner linings of bodily passages including epithelial and mucosal tissues). Such administration can be carried out using the active agents of the invention including pharmaceutically acceptable: salts, hydrates, or esters thereof, in lotions, creams, foams, patches, suspensions, solutions and/or suppositories (rectal and vaginal).

Transdermal administration can be accomplished through the use of a transdermal patch containing an active agent disclosed herein, and a carrier that can be inert to the active agent, can be non-toxic to the skin and can allow delivery of the active agent for systemic absorption into the blood stream via the skin. The carrier can take any number of forms such as creams and ointments, pastes, gels and occlusive devices. The creams and ointments can be viscous liquid or semisolid emulsions of either the oil-in-water or water-in-oil type. Pastes comprised of absorptive powders dispersed in petroleum or hydrophilic petroleum containing the active agent can also be suitable. A variety of occlusive devices can be used to release the active agent into the blood stream, such as a semi-permeable membrane covering a reservoir containing the active agent with or without a carrier, or a matrix containing the active agent. Other occlusive devices are known in the literature.

Compounds and/or compositions described herein can be administered rectally or vaginally in the form of a conventional suppository. Suppository formulations can be made from traditional materials, including cocoa butter, with or without the addition of waxes to alter the suppository's melting point and/or glycerin. Water-soluble suppository bases, such as polyethylene glycols of various molecular weights, can also be used.

Lipid formulations or nanocapsules can be used to introduce active agents into host cells either in vitro or in vivo. Lipid formulations and nanocapsules can be prepared by methods known in the art.

To increase the effectiveness of active agents, it can be desirable to combine an active agent with other agents effective in the treatment of the target disease. For example, other active agents effective in treating the target disease can be administered with the active agents. The other agents can be administered at the same time or at different times than the active agents disclosed herein.

Active agents of the invention can be useful for the treatment or inhibition of a pathological condition or disorder in a mammal, for example, a human patient. The invention accordingly provides methods of treating or inhibiting a pathological condition or disorder by providing to a mammal an active agent of the invention.

In certain embodiments, the method is effective to treat a condition associated with hyperproliferating cells, which typically results in a benign or malignant tumor. The method is particularly suitable for treating cancer.

Conditions associated with cell hyperproliferation include, but are not limited to cancer, psoriasis, actinic keratosis, Bowen's disease, keratoacanthoma, palmoplantar keratoderma, and epidermodysplasia verruciformis. These disorders involve uncontrolled cell division, leading to abnormal tissue growth and potential health complications.

Examples of cancers that are treatable by the method of the invention include, but are not limited to: bone cancer, pancreatic cancer, skin cancer, brain cancer, lung cancer, colorectal cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, multiple myeloma, solid tumors of childhood, lymphocytic lymphoma, cancer of the bladder (e.g., NMIBC), cancer of the kidney or urethra, carcinoma of the renal pelvis, neoplasm of the Central Nervous System (“CNS”), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

The dosages of the active agents can vary according to, for example, the particular use for which the treatment is made, the manner of administration of the compound, the health and condition of the patient and the judgment of the prescribing physician. The proportion or concentration of a compound of the invention in a pharmaceutical composition can vary depending upon a number of factors including: dosage, chemical characteristics (e.g.: hydrophobicity) and the route of administration. For example, the metal cation or metal complex and the at least one infectious agent can each be provided in an aqueous physiological buffer solution containing about 0.1 to about 10% w/v of the compound for parenteral administration. Some typical dose ranges are from approximately 1 g/kg to about 1 g/kg of body weight per day. In some embodiments, the dose range is from approximately 0.01 mg/kg to about 100 mg/kg of body weight per day. In certain embodiments, the dosage form comprises each active agent in amounts from about 0.001 mg to about 1000 mg or 0.01 mg to 100 mg or 0.1 mg to 10 mg. The dosage is likely to depend on such variables as the type and extent of progression of the disease or disorder, the overall health status of the particular patient, the relative biological efficacy of the compound selected, formulation of the excipient and its route of administration. Effective doses can be extrapolated from dose-response curves derived from in vitro or in vivo model test systems.

In certain embodiments, the at least one infectious agent will be administered systemically either prior to or after the metal cation administration (treatments vary from a few months to a few years). In certain embodiments, the at least one infectious agent is administered to the patient: locally, intravesically, intratumorally, intravenously or by other administration methods: daily, every 2 days, 3 times per week, every 3 days, weekly, biweekly, monthly, quarterly or some other administration schedule based on the patient's disease, stage and grade in an amount that ranges from the Maximum Recommended Starting Dose (“MRSD”) based on the Human Equivalent Dose (“HED”) to the Therapeutic Dose (“TD”) or Biologically Effective Dose (“BED”).

In certain embodiments, the metal complex will be administered locally and light activated (1 to 3 treatments) or will be administered systemically and activated via radiation (3 treatments per week over 2 to 6 weeks, with radiation daily Monday to Friday for the same time period). In certain embodiments, the metal complex is administered to the patient: locally, intravesically, intratumorally, intravenously or by other administration methods: daily, every 2 days, 3 times per week, every 3 days, weekly, biweekly, monthly, quarterly or some other administration schedule based on the patient's disease, stage and grade in an amount that ranges from the MRSD based on the HED to the TD or BED.

RDT is administered to the patient to activate at least one metal complex present to treat the condition. The term “radiation” as used herein encompasses non-ionizing radiation and ionizing radiation of the electromagnetic spectrum, including: infrared light, visible light, X-rays, Y-rays and quanta, and corpuscular radiation (i.e.: a-particles, p-particles, positrons, neutrons and heavy particles) capable of producing ions, in the less than 180 nanometers (“nm”) range. Suitable wavelengths of light activation; include, but are not limited to 180 to 1000 nm and most preferably 400 to 950 nm.

Radiation is directly ionizing if it carries an electric charge that directly interacts with atoms in the tissue or medium by electrostatic attraction. Indirect ionizing radiation is not electrically charged, but results in production of charged particles by which its energy is absorbed. It takes about 34 eV of energy to produce an ionization. Most human exposures to radiation are of energies of 0.05-5 Million electron Volts (MeV)—energies at which many ionizations occur as the radiation passes through cells. Most X-rays have a wavelength ranging from 0.001 to 10 nanometers. In the case of using a radio enhancer, a patient can be treated with a “diagnostic” dose of ionizing radiation, such as 0.02 Gray (“Gy”).

The radiation can be applied systemically or locally, topically or internally. The radiation is administered in a safe and effective dosage. For example, laser light is preferably administered in a dosage of at least 10 J/cm², preferably 10 or 100 J/cm² and more preferably from 25 to 90 J/cm². Radiation is preferably administered at a predetermined fluence rate or radiation dose to achieve the most desirable therapeutic effect—up to the highest permissible radiation dose, based on the patient's clinical status.

### Metal-Binding Glycoproteins

The metal complex is preferably administered to the patient along with a metal-binding glycoprotein. Metal-binding glycoproteins suitable for use in the invention are capable of binding transition metals and delivering to a biological target said metals and other materials complexed with said metals. The metal-binding glycoproteins are preferably capable of binding Group 8 metals and/or Group 9 metals, and most preferably Ru, Os and Rh. Most preferred are the iron-binding glycoproteins: transferrin, lactoferrin, ovotransferrin and melanotransferrin and variants thereof, with transferrin being most preferred. The glycoprotein can be purified from natural sources or can be from artificial sources. Thus, for example, the glycoprotein in certain embodiments is a recombinant transferrin, such as Apo Transferrin or OPTIFERRIN, a recombinant human transferrin available from InVitria, a division of Ventria Bioscience.

The invention will be illustrated in more detail with reference to the following examples, but it should be understood that the present invention is not deemed to be limited thereto.

### EXAMPLES

### Example 1—Cytotoxicity of BCG

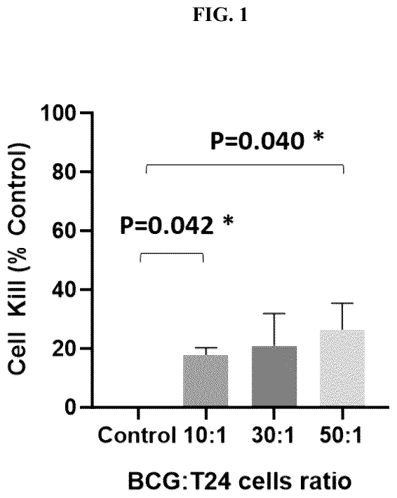

FIG. 1 shows the toxicity effect of BCG alone in T24 cells. The values are grouped along the increasing BCG:T24 ratio. Toxicity is presented as cell kill after 3 days of incubation, measured by Presto Blue viability assay. The values are normalized to Control (no BCG). Normalized cell kill is calculated as 100%−(Viability signal)/(Control Viability signal)*100%. Mean and SEM is shown for each group, N=2-5. Significance levels (P) are shown for one-sample T-tests (difference from 0% Control).

In T24 cells (human high grade bladder cancer), BCG vaccine (Russian strain, Verity Pharmaceuticals Inc.) exerted only weak to moderate cytotoxicity on its own, at BCG:T24 ratios ranging from 10:1 to 50:1, after 3 days of incubation and viability measurement by Presto Blue assay (FIG. 1). Similar studies detect toxic effect in T24 cells (measuring viability by MTT assay or cell loss) but at higher (100-5,000) BCG:T24 ratios (Kageyama et al., 1997; Chen et al., 2005; Yu et al., 2015).

### Example 2—RUVIDAR Binds to BCG Cells Producing RuBCG Formulation

Considering the beneficial outcome of overcoming the electrostatic barrier between the BCG and bladder cancer cells by switching BCG zeta potential from negative to positive, the binding of a cationic RUVIDAR complex to BCG cells was assessed.

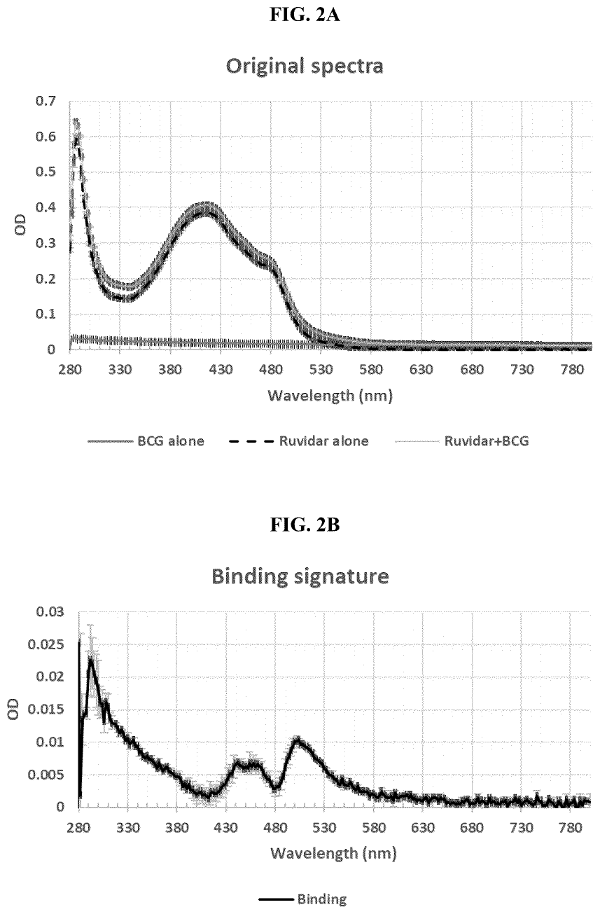

FIG. 2B shows the binding signature of RuBCG produced from 3.2 mg/mL BCG mixed with 1 mM RUVIDAR in water. Absorption spectra were measured (using Shimadzu UV 3600 spectrophotometer) at final concentration of 10 μM RUVIDAR (100-fold dilution of the original RuBCG mix). For reference, spectra of BCG alone (3.2 mg/mL) and RUVIDAR alone (1 mM) were used (FIG. 2A), also at 100-fold dilution of the original stocks. Binding signature is obtained by subtraction the spectra of BCG alone and RUVIDAR alone from the spectrum of RuBCG. For each wavelength, optical density (mean and SEM) is shown, N=2.

Spectral measurements indicate binding of RUVIDAR to BCG cells (RuBCG production) at 3.2 mg/mL BCG (3.2×10⁷ CFU/mL) and 1 mM RUVIDAR in water. Upon subtraction of the spectra of BCG alone and RUVIDAR alone from the RuBCG spectrum, the resulting absorbance surplus shows a characteristic pattern with peaks at 292, 442, and 504 nm, distinct from the spectral peaks of RUVIDAR alone (286 and 418 nm). RUVIDAR hence changes its absorbance spectral profile upon binding to BCG and producing RuBCG. Considering BCG concentration in the mix equal 3.2*10⁷ CFU/mL and 6.02*10¹⁷ RUVIDAR complexes/mL at 1 mM concentration, RUVIDAR:BCG stoichiometry is 1.88*10¹⁰, which is a dramatic excess of RUVIDAR complexes per BCG cells. Further BCG dilution (10-fold and greater) results in disappearance of the spectral signature of binding, likely because its magnitude falls below the detection threshold. This confirms indirectly that the binding event occurs on the surface of BCG cells: if there is too little cells in the mix, the spectral signal of binding is too weak to detect, despite the abundance of RUVIDAR in solution.

### Example 3—Charges of RuBCG and BCG

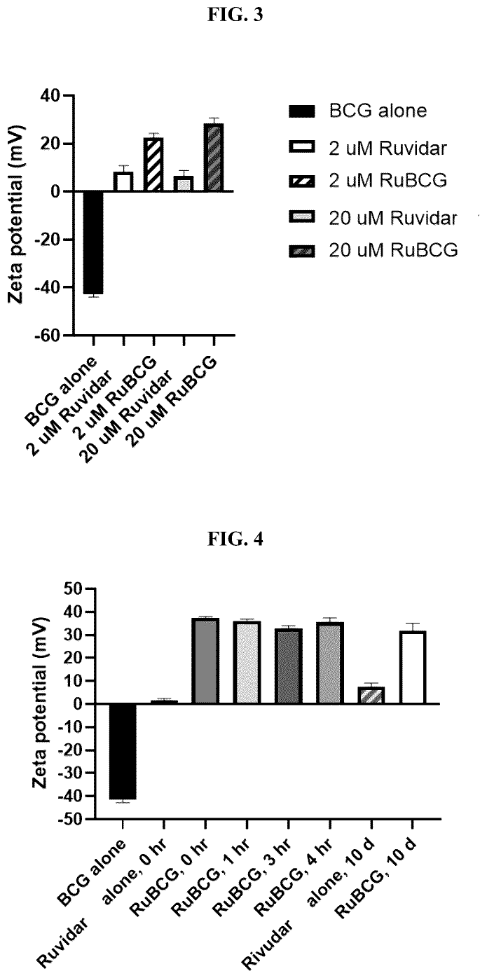

Referring to FIG. 3, Zeta potential of RuBCG prepared in water from 0.04 mg/mL BCG and 2 or 20 μM RUVIDAR. Zeta potential was measured using Zetasizer Ultra (Malvern). Zeta potentials of BCG alone and RUVIDAR alone were measured for comparison. Mean and SEM are shown, N=6-18.

While BCG alone, expectably, had a strongly negative zeta potential, RuBCG had zeta potential reversed to a strong positive one. This was observed at negligible to very weak zeta potential of RUVIDAR alone (because of low light scattering at the used concentrations). Intrinsic zeta charge of RUVIDAR (+36-40 mV when measured at 200-2,000 μM RUVIDAR) was not detected at 2-20 μM, despite a large excess of RUVIDAR vs. BCG cells. Therefore, only RuBCG zeta potential could be detected, with no interference from free RUVIDAR signal. RUVIDAR:BCG stoichiometric ratio during zeta potential measurements (3.0×10¹⁰ for 20 μM RUVIDAR) was only 1.6-fold higher than that for spectral measurements described above. Therefore, spectral and zeta potential measurements were performed at comparable RUVIDAR:BCG ratios. Zeta potential inversion suggests accumulation of RUVIDAR on BCG cells surface, so RUVIDAR intrinsic positive zeta potential starts manifesting itself, while not detectable in solution for unbound RUVIDAR. Most likely, this accumulation is due to binding of RUVIDAR to negatively charged teichoic acid (and probably other anionic groups) in the BCG cell wall, although accumulation within proteoglycans network also cannot be excluded.

### Example 4—Stability of BCG and RuBCG in Aqueous Solution

FIG. 4 shows the time course stability of zeta potential of RuBCG prepared in water from 0.04 mg/mL BCG and 20 μM RUVIDAR. The solutions were stored light protected at room temperature. Zeta potential was measured using Zetasizer Ultra (Malvern). Zeta potential of BCG alone and RUVIDAR alone was measured for comparison. Means and SEM are shown, N=3.

In clinical settings, reconstituted BCG vaccine must be used for instillation into the patient's bladder within 2 hours (if stored at +4° C.). At the same time, zeta potential of RuBCG is stable up to 10 days at room temperature storage, which indicates long-term stability of at least favorable electrostatic properties of the BCG cell wall.

### Example 5—RuBCG is More Cytotoxic than BCG

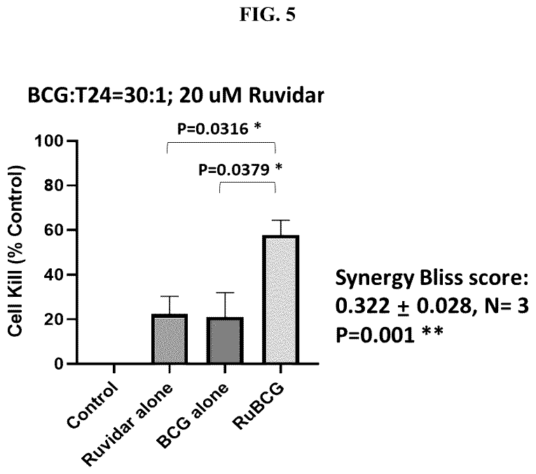

FIG. 5 shows synergistic toxicity of RuBCG in T24 cells. Toxicity is presented as cell kill after 3 days of incubation, measured by Presto Blue viability assay. The values are normalized to Control (no BCG, no RUVIDAR). Cell kill was calculated as 100%−(Viability signal)/(Control Viability signal)*100%. Synergy Bliss score was calculated as Viability at BCG alone*Viability at RUVIDAR alone−Viability at RuBCG. Mean and SEM are shown for each group, N=2-5. Significance levels (P) are shown for one-sample T-tests (difference from 0% Control).

In contrast to BCG alone, RuBCG has a strong cytotoxicity in T24 cells. Synergy Bliss score (calculated as Viability after BCG alone*Viability after RUVIDAR alone−Viability after RuBCG) showed a high value and was statistically significant. This indicates that RuBCG is more toxic for T24 cells than just a sum of BCG alone toxicity and RUVIDAR alone toxicity.

### Example 6—RUVIDAR-Based RuBCG (but not BCG) has Anti-Cancer Effect In Vivo: Prophetic Example

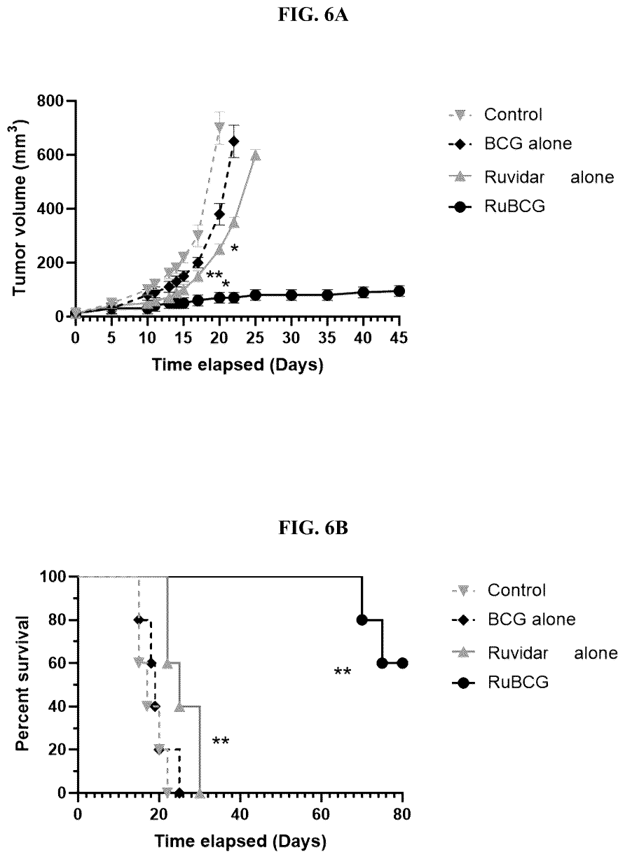

BCG alone injected into animals with established subcutaneous CT26.WT (mouse colon adenocarcinoma) tumors does not suppress tumor growth and does not improve survival (see FIGS. 6A and 6B). Injection of RUVIDAR results in tumor growth suppression and significantly increases survival, but the effect size is small. In contrast, RuBCG injection results in a strong suppression of the tumor growth and increase in survival, much more than could be expected from just combination of the effects of BCG alone and RUVIDAR alone. This demonstrates anti-tumor efficacy of RuBCG not only in cells in vitro but in tumor in vivo.

FIGS. 6A and 6B show in vivo efficacy of RuBCG. CT26.WT (human colon adenocarcinoma) tumors are induced subcutaneously in male BALB/C mice. Untreated animals are used as a control. BCG alone and RUVIDAR groups are used for comparison with RuBCG group. FIG. 6A shows tumor volume growth (means and SEM are shown). FIG. 6B shows Kaplan-Meier analysis of survival curves. N=5 in each group. Significance levels (vs. Control): * P<0.05; ** P<0.01.

Subcutaneous CT26.WT colorectal tumors are induced by subcutaneous injection of CT26.WT cells (350,000 cells per mouse in 100 μL PBS) in male BALB/C mice. The animals are monitored for tumor growth by measuring in two dimensions (width and length). Tumor volume is estimated as V=π/6×Length×Width². When the tumors measure 5 mm in any dimension, IV injections are performed into tail vein (100 μL per 20 g mouse):

1. BCG alone (10⁶ CFU/animal). BCG suspension is prepared by 40-fold dilution of the 40 mg/mL BCG stock in 20% Propylene glycol (PG) in 5% Dextrose solution (25 uL of BCG stock per 1 mL of solvent).

2. RUVIDAR alone (1 mg/kg of body weight). RUVIDAR solution is prepared as 0.2 mg/mL RUVIDAR in 20% Propylene glycol in 5% Dextrose solution.

3. RuBCG (1 mg/kg RUVIDAR+10⁶ CFU BCG). RuBCG formulation is prepared as 0.2 mg/mL RUVIDAR in 20% Propylene glycol in 5% Dextrose solution+25 μL of 40 mg/mL BCG stock per 1 mL of RuBCG.

The tumors are monitored regularly. When any of the tumor dimensions exceed 1 cm or the tumor has significant ulceration, the animals are considered reaching endpoint and sacrificed.

### Example 7—RuBCG is More Immunogenic than BCG

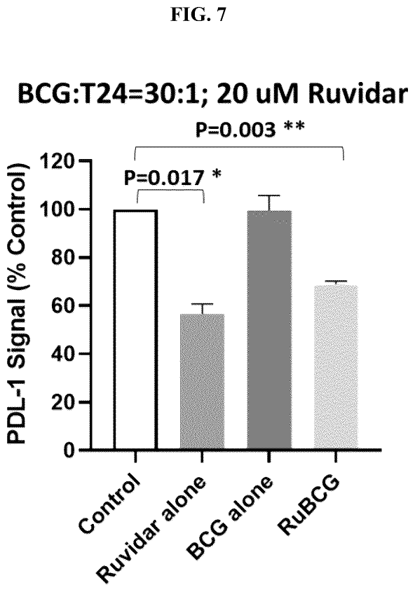

In addition to increased toxicity, RuBCG downregulated signals of immune checkpoint inhibitors PDL-1 (FIG. 7) suggesting immunogenic effect. RuBCG exceeds the effect by RUVIDAR alone. Together with the synergistic cytotoxicity effect, the data indicate RuBCG is capable to induce both immunogenic and cytotoxic effects, which are not attainable using BCG alone.

### Example 8—RUTHERRIN-Based RuBCG (but not BCG) has Anti-Cancer Effect In Vivo: Prophetic Example

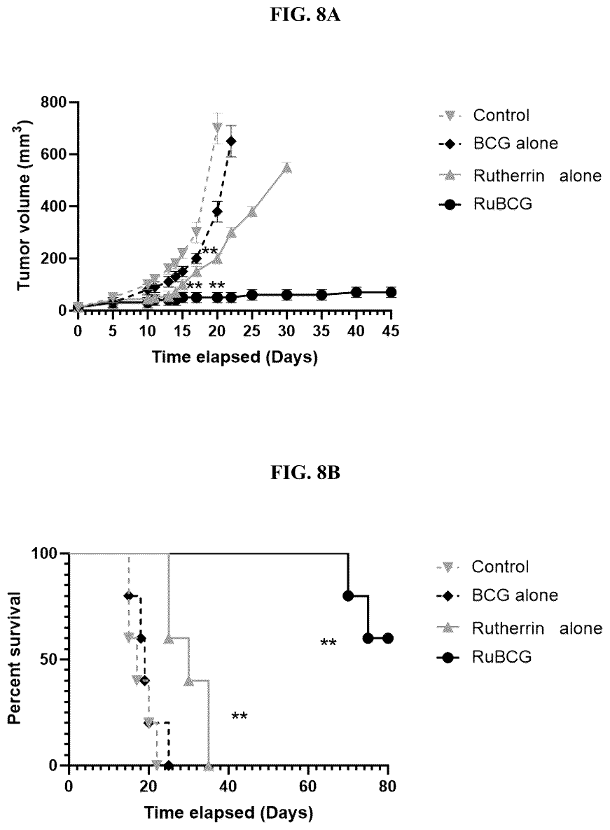

BCG alone injected to animals with established subcutaneous CT26.WT (mouse colon adenocarcinoma) tumors does not suppress tumor growth and does not improve survival (FIGS. 8A and 8B). Injection of RUTHERRIN results in tumor growth suppression and significantly increases animals survival, but the effect size was small. In contrast, RuBCG injection results in a strong suppression of tumor growth and increase in survival, much more than could be expected from just combination of the effects of BCG alone and RUTHERRIN alone. This demonstrated anti-tumor efficacy of RuBCG not only in cells in vitro but in tumor in vivo.

In vivo efficacy of RUTHERRIN-based RuBCG. CT26.WT (human colon adenocarcinoma) tumors are induced subcutaneously in male BALB/C mice. Untreated animals are used as a control. BCG alone and RUTHERRIN groups are used for comparison with RuBCG group. FIG. 8A shows tumor volume growth (means and SEM are shown). FIG. 8B shows Kaplan-Meier analysis of survival curves. N=5 in each group. Significance levels (vs. Control): * P<0.05; ** P<0.01.

Subcutaneous CT26.WT colorectal tumors are induced by subcutaneous injection of CT26.WT cells (350,000 cells per mouse in 100 L PBS) in male BALB/C mice. The animals are monitored for tumors growth by measuring in two dimensions (width and length). Tumor volume is estimated as V=7c/6*Length*Width². When the tumors measure 5 mm in any dimension, IV injections are performed into tail vein (100 L per 20 g mouse):

1. BCG alone (10⁶ CFU/animal). BCG suspension is prepared by 40-fold dilution of the 40 mg/mL BCG stock in 20% Propylene glycol (PG) in 5% Dextrose solution (25 uL of BCG stock per 1 mL of solvent).

2. RUTHERRIN alone (1 mg/kg of body weight). RUTHERRIN solution is prepared as 0.2 mg/mL Ruvidar™+4.54 mg/mL Optiferrin® in 20% Propylene glycol in 5% Dextrose solution.

3. RuBCG (1 mg/kg Ruvidar™+10⁶ CFU BCG). RuBCG formulation is prepared as 0.2 mg/mL Ruvidar™+4.54 mg/mL Optiferrin® in 20% Propylene glycol in 5% Dextrose solution+25 μL of 40 mg/mL BCG stock per 1 mL of RuBCG.

The tumors are monitored regularly. When any of the tumor dimensions exceed 1 cm or the tumor has significant ulceration, the animals are considered reaching endpoint and sacrificed.

### Example 8—Binding of Iron Ions to BCG: Zeta Potential

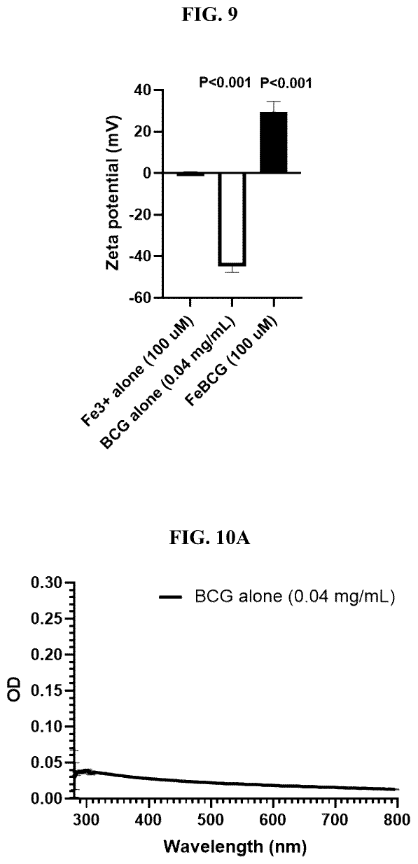

BCG cells were diluted in water to 0.04 mg/mL, and Fe³⁺ ions (in a form of 100 uM Fe(NO₃)₃) were added. Zeta potential was measured immediately using a Zeta Sizer Ultra (Malvern). Zeta potential of BCG alone and Fe³⁺ alone was also measured. As shown in FIG. 9, a change in zeta potential of BCG cells occurred upon mixing with Fe³⁺. The intrinsic negative zeta potential of BCG cells was switched to positive in BCG+Fe³⁺ mix. Zeta potential of the free Fe³⁺ was not detectable at 100 uM. Therefore, the inversion of Zeta potential cannot be explained just by the presence of free Fe³⁺ ions and can be attributed instead to Fe³⁺ binding to BCG surface.

### Example 9—Binding of Iron Ions to BCG: Spectral Measurements

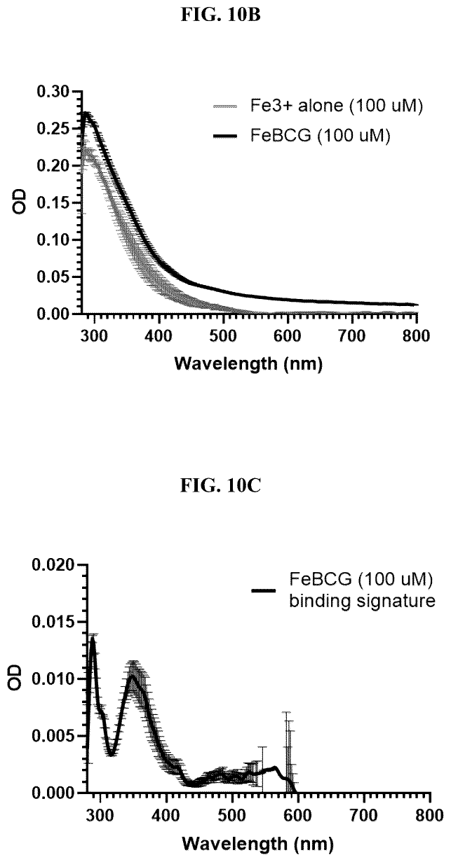

BCG (0.04 mg/mL) was mixed with Fe³⁺ ions (in a form of 100 uM Fe(NO₃)₃), and an absorption spectrum was measured (FIG. 10A). The absorbance of BCG alone (FIG. 10B) was also measured (to account for bacterial cells turbidity factor), as well as the spectrum of free 100 uM Fe³⁺ (FIG. 10C). Both of these spectra were subtracted from the Fe³⁺+BCG mix spectrum, to obtain the binding signature as evidence of Fe³⁺ and BCG interaction: formation of FeBCG due to binding of Fe³⁺ ions to the BCG cell surface. Fe³⁺+BCG mix showed characteristic binding signature with few peaks: MEC=135±3 M−1cm−1 at 288 nm and 102±13 M−1cm−1 at 348 nm (the numbers represent Mean±SEM).

### Example 10—In Vitro Efficacy of FeBCG

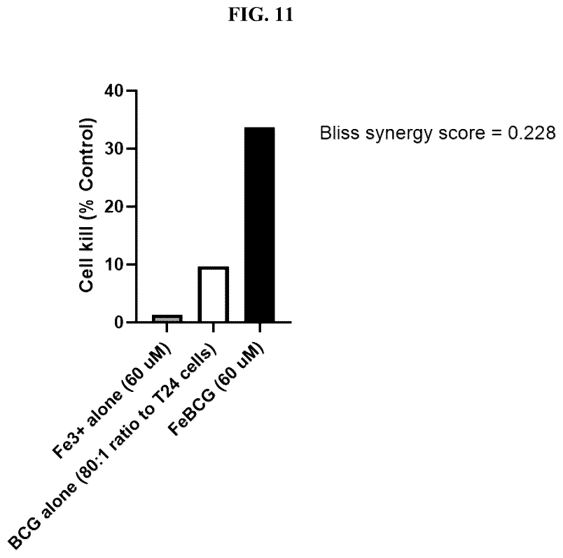

Cells at a density of 4,000 T24 cells/well (in a 96-well plate, 200 μL of FBS-supplemented DMEM in each well) were incubated with 80-fold excess of BCG cells and 100 uM Fe³⁺ (as Fe(NO₃)₂) for four days. After that, cell viability was measured using Presto Blue viability assay, and cell viability was calculated as the Presto Blue signal relative to the Control (no BCG, no Fe³⁺). See FIG. 11. Cell kill percent was calculated as 100%−Viability*100%. Also, synergistic effect of BCG and Fe³⁺ was assessed by calculation of Bliss synergy score: Viability after BCG exposure+Viability after Fe³⁺ exposure−Viability after FeBCG exposure

The positive value of the Bliss score (Bliss>0) denotes synergistic effect of FeBCG vs. BCG and Fe³⁺ alone.

The data indicate synergistic effect after exposure of T24 cells to FeBCG (33.7% kill, Bliss synergy score=0.228), at relatively low toxicity of BCG alone (9.7% kill) and negligible toxicity of Fe³⁺ alone (1.4% kill).

### ADDITIONAL NUMBERED EMBODIMENTS OF THE INVENTION

1. A composition for treating a condition associated with hyperproliferation of cells, comprising:
at least one infectious agent selected from the group consisting of bacteria, protozoa, fungi, viruses, algae and structural components thereof, wherein the at least one infectious agent has a net negative charge and is effective to treat the condition; and
at least one metal cation in an amount effective to more than offset the net negative charge of the at least one infectious agent such that the composition has a net positive charge.

2. The composition of embodiment 1, wherein the at least one metal cation is contained in at least one metal complex represented by formula (I):

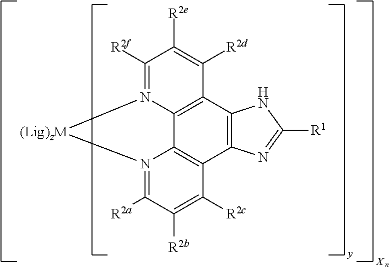

including hydrates, solvates, pharmaceutically acceptable salts and prodrugs thereof, wherein:
the at least one metal cation (M) is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
y=1,2, or 3;
z=0, 1, or 2;
Lig at each occurrence is independently selected from the group consisting of

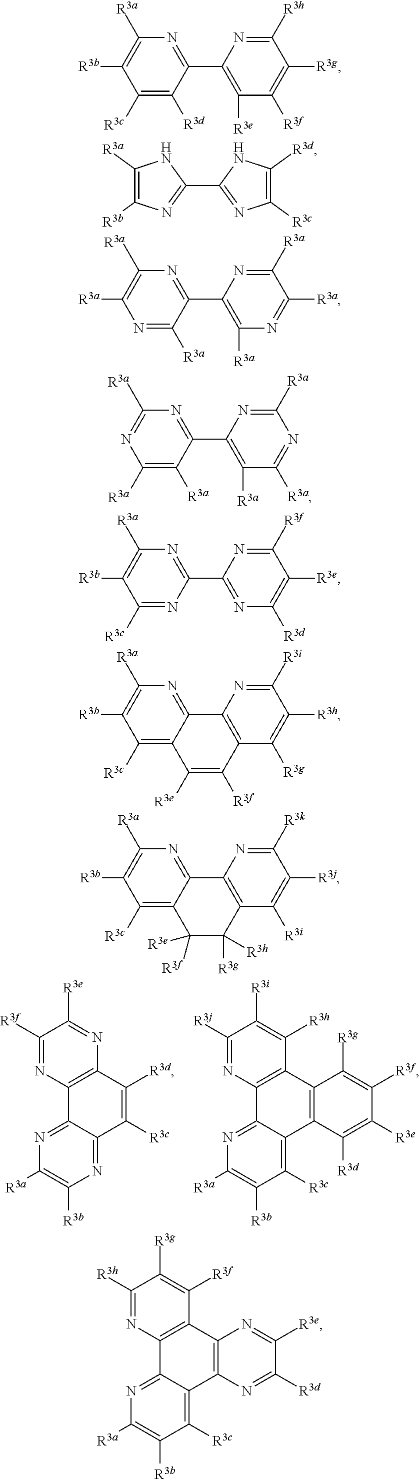

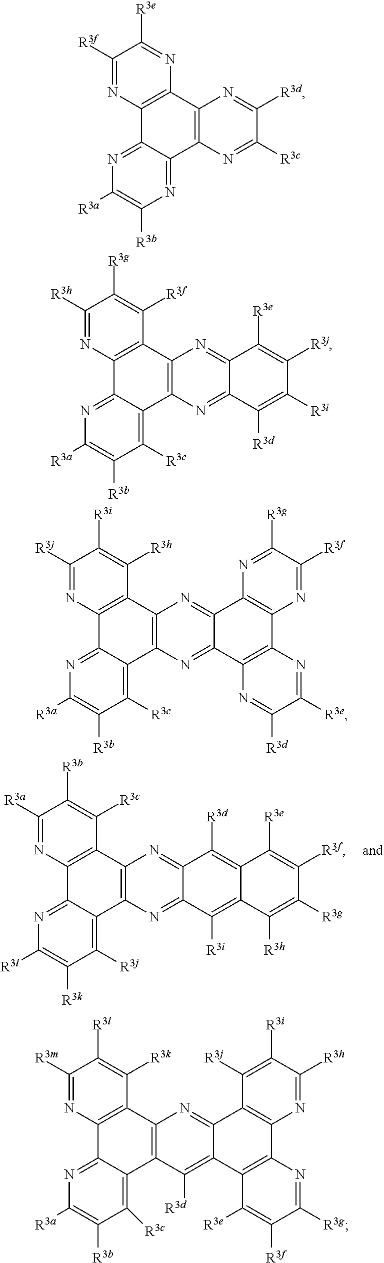

R¹ is selected from the group consisting of

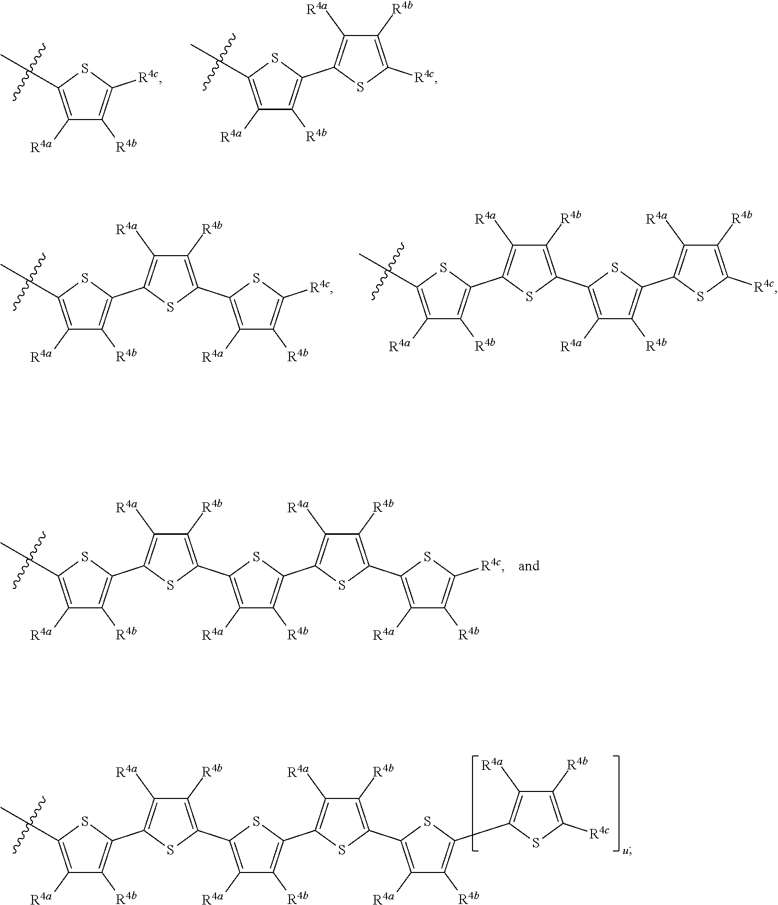

u is an integer of 1 to 20;
R^{2a}, R^{2b}, R^{2c}, R^{2d}, R^{2e}, and R^{2f} at each occurrence are each independently selected from the group consisting of hydrogen, C1-6 optionally substituted alkyl, C1-6 optionally substituted branched alkyl, C3-7 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h}, R³ⁱ, R^{3j}, R^{3k}, R^{3l}, and R^{3m} at each occurrence are each independently selected from the group consisting of hydrogen, C1-6 optionally substituted alkyl, C1-6 optionally substituted branched alkyl, C1-6 optionally substituted haloalkyl, C1-6 optionally substituted alkoxy, and CO₂R⁸;
R^{4a}, R^{4b}, and R^{4c} at each occurrence are each independently selected from the group consisting of hydrogen, C1-6 optionally substituted alkyl, C1-6 optionally substituted branched alkyl, C1-6 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;
R⁶ at each occurrence is independently selected from the group consisting of hydrogen and optionally substituted alkyl;
R⁷ at each occurrence is independently selected from the group consisting of hydrogen and optionally substituted alkyl; and
R⁸ at each occurrence is independently selected from the group consisting of hydrogen and optionally substituted alkyl.

3. The composition of embodiment 2, comprising complexes of the at least one infectious agent and the at least one metal complex.

4. The composition of any one of embodiments 1-3, further comprising transferrin.

5. The composition of any one of embodiments 2-4, wherein the at least one metal complex comprises at least one member selected from the group consisting of:
Ru(2,2′-bipyridine)₂(2-(2′,2″:5″,2′″-terthiophene)-imidazo[4,5-]A[1,10]phenanthroline);
Ru(2,2′-bipyridine)₂(2-(2′,2″:5″,2′″;5′″,2″″-quaterthiophene)-imidazo[4,5-f][1,10]phenanthroline);
Ru(4,4′-dimethyl-2,2′-bipyridine)₂(2-thiophenimidazo[4,5-f][1,10]phenanthroline);
Ru(4,4′-dimethyl-2,2′-bipyridine)₂(2-(2′,2″-bithiophene)-imidazo[4,5-f][1,10]phenanthroline);
Ru(4,4′-dimethyl-2,2′-bipyridine)₂(2-(2′,2″:5″,2′″-terthiophene)-imidazo[4,5-f][1,10]phenanthroline);
Ru(4,4′-dimethyl-2,2′-bipyridine)₂(2-(2′,2″:5″,2′″;5′″,2″″-quaterthiophene)-imidazo[4,5-f][1,10]phenanthroline);
Ru(4,4′-di-t-butyl-2,2′-bipyridine)₂(2-thiophenimidazo[4,5-f][1,10]phenanthroline);
Ru(4,4′-di-t-butyl-2,2′-bipyridine)₂(2-(2′,2″-bithiophene)-imidazo[4,5-f][1,10]phenanthroline);
Ru(4,4′-di-t-butyl-2,2′-bipyridine)₂(2-(2′,2″:5″,2′″-terthiophene)-imidazo[4,5-f][1,10]phenanthroline);
Ru(4,4′-dimethoxy-2,2′-bipyridine)₂(2-(2′,2″:5″,2′″-terthiophene)-imidazo[4,5-f][1,10]phenanthroline);
Ru(5,5′-dimethyl-2,2′-bipyridine)₂(2-thiophenimidazo[4,5-f][1,10]phenanthroline);
Ru(5,5′-dimethyl-2,2′-bipyridine)₂(2-(2′,2″-bithiophene)-imidazo[4,5-f][1,10]phenanthroline);
Ru(5,5′-dimethyl-2,2′-bipyridine)₂(2-(2′,2″:5″,2′″-terthiophene)-imidazo[4,5-f][1,10]phenanthroline);
Ru(6,6′-dimethyl-2,2′-bipyridine)₂(2-thiophenimidazo[4,5-f][1,10]phenanthroline);
Ru(6,6′-dimethyl-2,2′-bipyridine)₂(2-(2′,2″-bithiophene)-imidazo[4,5-f][1,10]phenanthroline);
Ru(6,6′-dimethyl-2,2′-bipyridine)₂(2-(2′,2″:5″,2′″-terthiophene)-imidazo[4,5-f][1,10]phenanthroline);
Ru(4,4′-di(methylcarboxy)-2,2′-bipyridine)₂(2-(2′,2′″:5″,2′″-terthiophene)-imidazo[4,5-f][1,10]phenanthroline);
Ru(2,2′-bipyrimidine)₂(2-(2′,2″:5″,2′″-terthiophene)-imidazo[4,5-f][1,10]phenanthroline);
Ru(2,2′-bipyrimidine)(4,4′-dimethyl-2,2′-bipyridine)(2-(2′,2′″:5″,2′″-terthiophene)-imidazo[4,5-f][1,10]phenanthroline);
Ru(1,10-phenanthroline)₂(2-thiophenimidazo[4,5-f][1,10]phenanthroline);
Os(2,2′-bipyridine)₂(2-thiophenimidazo[4,5-f][1,10]phenanthroline);
Os(2,2′-bipyridine)₂(2-(2′,2″-bithiophene)-imidazo[4,5-][1,10]phenanthroline);
Os(2,2′-bipyridine)₂(2-(2′,2″:5″,2′″-terthiophene)-imidazo[4,5-f][1,10]phenanthroline);
Os(1,10-phenanthroline)₂(2-thiophenimidazo[4,5-f][1,10]phenanthroline);
Os(1,10-phenanthroline)₂(2-(2′,2″-bithiophene)-imidazo[4,5-f][1,10]phenanthroline);
Os(1,10-phenanthroline)₂(2-(2′,2″:5″,2′″-terthiophene)-imidazo[4,5-f][1,10]phenanthroline);
Os(4,4′-dimethyl-2,2′-bipyridine)₂(2-thiophenimidazo[4,5-f][1,10]phenanthroline);
Os(4,4′-dimethyl-2,2′-bipyridine)₂(2-(2′,2″-bithiophene)-imidazo[4,5-f][1,10]phenanthroline);
Os(4,4′-dimethyl-2,2′-bipyridine)₂(2-(2′,2′″:5″,2′″-terthiophene)-imidazo[4,5-f][1,10]phenanthroline); and
pharmaceutically acceptable salts thereof.

6. The composition of any one of embodiments 2-4, wherein the at least one metal complex is Ru(4,4′-dimethyl-2,2′-bipyridine)₂(2-(2′,2″:5″,2′″-terthiophene)-imidazo[4,5-f][1,10]phenanthroline) or a pharmaceutically acceptable salt thereof.

7. The composition of any one of embodiments 2-6, wherein a single dose of the composition contains 0.001 μg to 80000 μg of the at least one metal complex and 1×10⁻⁴ to 8×10⁻⁸ CFU of the at least one infectious agent.

8. The composition of any one of embodiments 1-7, further comprising at least one negatively charged substance additional to the at least one infectious agent, wherein the negatively charged substance is at least one of a lipopolysaccharide and a negatively charged microorganism.

9. The composition of any one of embodiments 1-8, wherein the at least one infectious agent comprises engineered bacteria selected from the group consisting of live attenuated Mycobacterium Bovis, engineered Salmonella, engineered Staphylococcus, engineered Listeria, engineered E-coli, engineered Bifidobacterium and engineered Clostridium.

10. The composition of any one of embodiments 1-9, comprising at least two different infectious agents selected from the group consisting of bacteria, protozoa, fungi, viruses, algae and structural components thereof.

11. A method for treating a condition associated with hyperproliferation of cells in a patient, comprising the steps:
(a) administering to the patient at least one infectious agent selected from the group consisting of bacteria, protozoa, fungi, viruses and algae, wherein the at least one infectious agent has a net negative charge and is effective to treat the condition; and
(b) administering to the patient before, during and/or after step (a) at least one metal cation, wherein the method is effective to slow cell hyperproliferation, slow tumor growth and/or reduce tumor volume.

12. A method for treating a condition associated with hyperproliferation of cells in a patient, comprising administering to the patient a composition of embodiment 1 in an amount and for a duration effective to slow cell hyperproliferation, slow tumor growth and/or reduce tumor volume.

13. The method of embodiment 12, wherein the at least one metal cation is contained in at least one metal complex represented by formula (I):

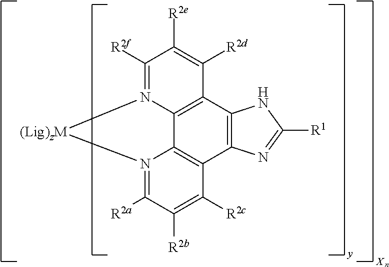

including hydrates, solvates, pharmaceutically acceptable salts and prodrugs thereof, wherein:
the at least one metal cation (M) is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
y=1,2, or 3;
z=0, 1, or 2;
Lig at each occurrence is independently selected from the group consisting of

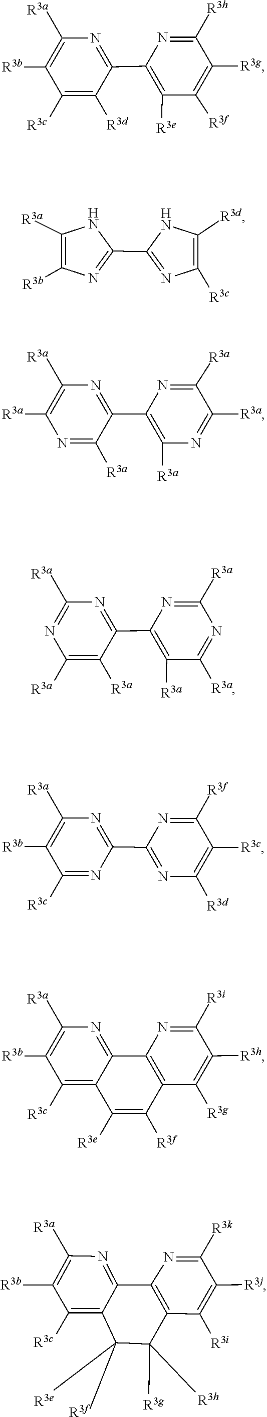

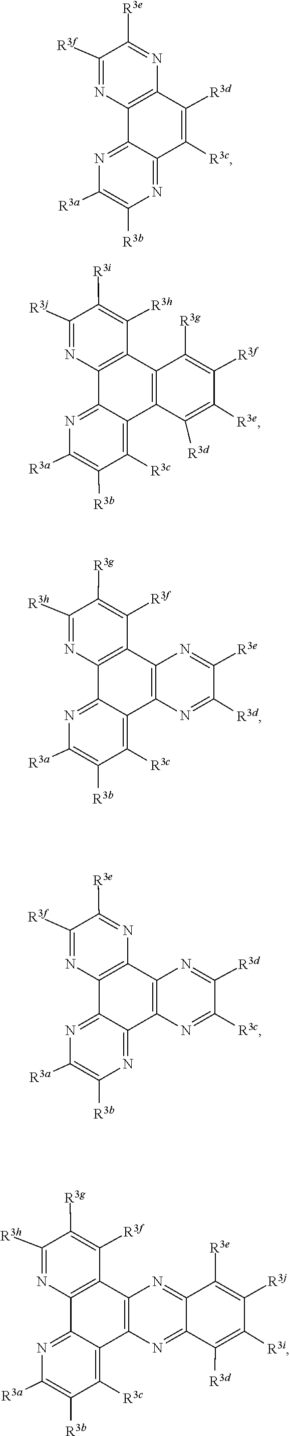

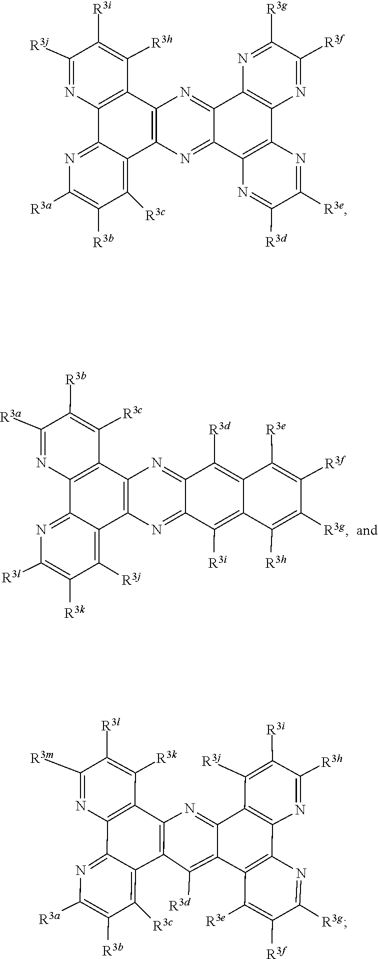

R¹ is selected from the group consisting of

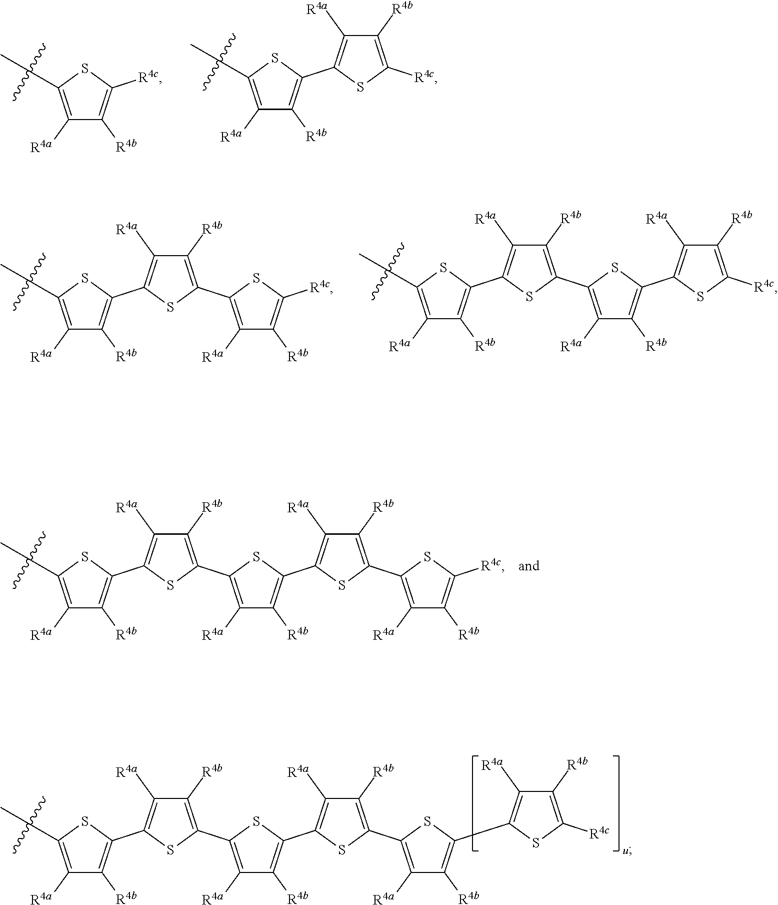

u is an integer of 1 to 20;
R^{2a}, R^{2b}, R^{2c}, R^{2d}, R^{2e}, and R^{2f} at each occurrence are each independently selected from the group consisting of hydrogen, C1-6 optionally substituted alkyl, C1-6 optionally substituted branched alkyl, C3-7 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h}, R³ⁱ, R^{3j}, R^{3k}, R^{3l}, and R^{3m} at each occurrence are each independently selected from the group consisting of hydrogen, C1-6 optionally substituted alkyl, C1-6 optionally substituted branched alkyl, C1-6 optionally substituted haloalkyl, C1-6 optionally substituted alkoxy, and CO₂R⁸;
R^{4a}, R^{4b}, and R^{4c} at each occurrence are each independently selected from the group consisting of hydrogen, C1-6 optionally substituted alkyl, C1-6 optionally substituted branched alkyl, C1-6 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;
R⁶ at each occurrence is independently selected from the group consisting of hydrogen and optionally substituted alkyl;
R⁷ at each occurrence is independently selected from the group consisting of hydrogen and optionally substituted alkyl; and
R⁸ at each occurrence is independently selected from the group consisting of hydrogen and optionally substituted alkyl.

14. The method of embodiment 13, wherein the composition is formed in vivo by administering the at least one metal complex and the at least one infectious agent separately.

15. The method of embodiment 12 or 13, wherein the composition is formed prior to being administered to the patient.

16. The method of any one of embodiments 12-15, wherein the condition is non-muscle invasive bladder cancer.

17. The method of any one of embodiments 12-16, further comprising administering transferrin to the patient.

18. The method of any one of embodiments 13-17, wherein the at least one metal complex comprises at least one member selected from the group consisting of:
Ru(2,2′-bipyridine)₂(2-(2′,2″:5″,2′″-terthiophene)-imidazo[4,5-f][1,10]phenanthroline);
Ru(2,2′-bipyridine)₂(2-(2′,2″:5″,2′″;5′″,2″″-quaterthiophene)-imidazo[4,5-f][1,10]phenanthroline);
Ru(4,4′-dimethyl-2,2′-bipyridine)₂(2-thiophenimidazo[4,5-f][1,10]phenanthroline);
Ru(4,4′-dimethyl-2,2′-bipyridine)₂(2-(2′,2″-bithiophene)-imidazo[4,5-f][1,10]phenanthroline);
Ru(4,4′-dimethyl-2,2′-bipyridine)₂(2-(2′,2″:5″,2′″-terthiophene)-imidazo[4,5-f][1,10]phenanthroline);
Ru(4,4′-dimethyl-2,2′-bipyridine)₂(2-(2′,2″:5″,2′″;5′″,2″″-quaterthiophene)-imidazo[4,5-f][1,10]phenanthroline);
Ru(4,4′-di-t-butyl-2,2′-bipyridine)₂(2-thiophenimidazo[4,5-][1,10]phenanthroline);
Ru(4,4′-di-t-butyl-2,2′-bipyridine)₂(2-(2′,2″-bithiophene)-imidazo[4,5-f][1,10]phenanthroline);
Ru(4,4′-di-t-butyl-2,2′-bipyridine)₂(2-(2′,2″:5″,2′″-terthiophene)-imidazo[4,5-f][1,10]phenanthroline);
Ru(4,4′-dimethoxy-2,2′-bipyridine)₂(2-(2′,2″:5″,2′″-terthiophene)-imidazo[4,5-f][1,10]phenanthroline);
Ru(5,5′-dimethyl-2,2′-bipyridine)₂(2-thiophenimidazo[4,5-][1,10]phenanthroline);
Ru(5,5′-dimethyl-2,2′-bipyridine)₂(2-(2′,2″-bithiophene)-imidazo[4,5-f][1,10]phenanthroline);
Ru(5,5′-dimethyl-2,2′-bipyridine)₂(2-(2′,2″:5″,2′″-terthiophene)-imidazo[4,5-f][1,10]phenanthroline);
Ru(6,6′-dimethyl-2,2′-bipyridine)₂(2-thiophenimidazo[4,5-][1,10]phenanthroline);
Ru(6,6′-dimethyl-2,2′-bipyridine)₂(2-(2′,2″-bithiophene)-imidazo[4,5-f][1,10]phenanthroline);
Ru(6,6′-dimethyl-2,2′-bipyridine)₂(2-(2′,2″:5″,2′″-terthiophene)-imidazo[4,5-f][1,10]phenanthroline);
Ru(4,4′-di(methylcarboxy)-2,2′-bipyridine)₂(2-(2′,2′″:5″,2′″-terthiophene)-imidazo[4,5-f][1,10]phenanthroline);
Ru(2,2′-bipyrimidine)₂(2-(2′,2″:5″,2′″-terthiophene)-imidazo[4,5-f][1,10]phenanthroline);
Ru(2,2′-bipyrimidine)(4,4′-dimethyl-2,2′-bipyridine)(2-(2′,2′″:5″,2′″-terthiophene)-imidazo[4,5-f][1,10]phenanthroline);
Ru(1,10-phenanthroline)₂(2-thiophenimidazo[4,5-f][1,10]phenanthroline);
Os(2,2′-bipyridine)₂(2-thiophenimidazo[4,5-f][1,10]phenanthroline);
Os(2,2′-bipyridine)₂(2-(2′,2″-bithiophene)-imidazo[4,5-f][1,10]phenanthroline);
Os(2,2′-bipyridine)₂(2-(2′,2″:5″,2′″-terthiophene)-imidazo[4,5-f][1,10]phenanthroline);
Os(1,10-phenanthroline)₂(2-thiophenimidazo[4,5-f][1,10]phenanthroline);
Os(1,10-phenanthroline)₂(2-(2′,2″-bithiophene)-imidazo[4,5-f][1,10]phenanthroline);
Os(1,10-phenanthroline)₂(2-(2′,2″:5″,2′″-terthiophene)-imidazo[4,5-][1,10]phenanthroline);
Os(4,4′-dimethyl-2,2′-bipyridine)₂(2-thiophenimidazo[4,5-][1,10]phenanthroline);
Os(4,4′-dimethyl-2,2′-bipyridine)₂(2-(2′,2″-bithiophene)-imidazo[4,5-][1,10]phenanthroline);
Os(4,4′-dimethyl-2,2′-bipyridine)₂(2-(2′,2′″:5″,2′″-terthiophene)-imidazo[4,5-f][1,10]phenanthroline); and
pharmaceutically acceptable salts thereof.

19. The method of any one of embodiments 13-17, wherein the at least one metal complex is Ru(4,4′-dimethyl-2,2′-bipyridine)₂(2-(2′,2″:5″,2′″-terthiophene)-imidazo[4,5-f][1,10]phenanthroline) or a pharmaceutically acceptable salt thereof.

20. The method of any one of embodiments 12-19, wherein a single dose of the composition contains 0.001 μg to 80000 μg of the at least one metal cation and 1×10⁻⁴ to 8×10⁻⁸ CFU of the at least one infectious agent.

21. The method of any one of embodiments 12-20, wherein the at least one infectious agent comprises engineered bacteria selected from the group consisting of live attenuated Mycobacterium Bovis, engineered Salmonella, engineered Staphylococcus, engineered Listeria, engineered E-coli, engineered Bifidobacterium, and engineered Clostridium.

22. The method of any one of embodiments 12-21, further comprising administering at least one negatively charged substance additional to the at least one infectious agent, wherein the negatively charged substance is at least one of a lipopolysaccharide and a negatively charged microorganism.

23. The method of any one of embodiments 12-22, wherein the administering step comprises administering to the patient at least two different infectious agents selected from the group consisting of bacteria, protozoa, fungi, viruses, algae and structural components thereof.

While the invention has been described in detail and with reference to specific examples thereof, it will be apparent to one skilled in the art that various changes and modifications can be made therein without departing from the spirit and scope thereof.

### REFERENCES CITED

1. Behrend. Patients with primary brain tumors. Oncology Nursing Forum. 2014; 41(3):335-336. doi: 10.1188/14.Onf.335-336
2. Berneis et al. The enhancement of the after effect of ionizing radiation by a cytotoxic methylhydrazine derivative. European Journal of Cancer. 2004; 40(13):1928-1933. doi: 10.1016/j.ejca.2004.04.013.
3. Bevers R F, de Boer E C, Kurth K H, Schamhart D H. BCG-induced interleukin-6 upregulation and BCG internalization in well and poorly differentiated human bladder cancer cell lines. Comparative Study Eur Cytokine Netw. 1998 June; 9(2):181-6.
4. Bevers R F M, Kurth K-H, Schamhart DHJ. Role of urothelial cells in BCG immunotherapy for superficial bladder cancer. Review Br J Cancer. 2004 Aug. 16; 91(4):607-12. doi: 10.1038/sj.bjc.6602026.
5. Bielski et al. Highlights of current research involving superoxide and perhydroxyl radicals in aqueous solutions. International Journal of Radiation Biology. 2009; 59(2):291-319. doi: 10.1080/09553009114550301.
6. Cardone R A, Casavola V, Reshkin S J. The role of disturbed pH dynamics and the Na+/H+ exchanger in metastasis. Nat Rev Cancer 2005; 5:786-95.
7. Chen F, Zhang G, Iwamoto Y, See W A. BCG directly induces cell cycle arrest in human transitional carcinoma cell lines as a consequence of integrin cross-linking. BMC Urol. 2005 May 12:5:8. doi: 10.1186/1471-2490-5-8.
8. Cherenkov. (1934). “Visible emission of clean liquids by action of γ radiation”. Doklady Akademii Nauk SSSR. 2: 451. Reprinted in Selected Papers of Soviet Physicists, Usp. Fiz. Nauk 93 (1967) 385. V sbornike: Pavel Alekseyevich Cerenkov: Chelovek i Otkrytie pod redaktsiej A. N. Gorbunova i E. P. Cerenkovoj, M., Nauka, 1999, s. 149-153. (ref Archived Oct. 22, 2007, at the Wayback Machine.
9. Costantini E, Lazzeri M, Porena M. GAGs and GAGs diseases: when pathophysiology supports the clinic. Review Urologia. 2013 July-September; 80(3):173-8. doi: 10.5301/RU.2013.11500. Epub 2013 Sep. 30.
10. Davis J A, Hauser P, Kyker K, Hurst R E, Culkin D J. Urothelial Proteoglycans of the Normal Human and Interstitial Cystitis Bladder. The Journal of Urology, April 2006, 175(4):93. DOI: 10.1016/S0022-5347(18)32547-3.
11. De Guillebon et al. (2020), Beyond the concept of cold and hot tumors for the development of novel predictive biomarkers and the rational design of immunotherapy combination. Int. J. Cancer, 147: 1509-1518. https://doi.org/10.1002/ijc.32889.
12. Dobrzynska I, Szachowicz-Petelska B, Darewicz B, Figaszewski Z A. Characterization of human bladder cell membrane during cancer transformation. J Membr Biol. 2015 April; 248(2):301-7. doi: 10.1007/s00232-015-9770-4. Epub 2015 Jan. 9.
13. Durek C, Brandau S, Ulmer A J, Flad H D, Jocham D, Böhle A. Bacillus-Calmette-Guérin (BCG) and 3D tumors: an in vitro model for the study of adhesion and invasion. J Urol, 1999, 162: 600-605.
14. Fan, J. Y., Huang, Y., Li, Y., Muluh, T. A., Fu, S. Z., & Wu, J. B. (2022). Bacteria in cancer therapy: A new generation of weapons. Cancer Medicine, 11(23), 4457-4468.
15. Galano, R. J. Reiter, Melatonin and its metabolites versus oxidative stress: from individual actions to collective protection, J. Pineal Res. 65 (1) (2018) e12514.
16. Gao et al. The Mechanisms of Ferroptosis Under Hypoxia. Cell Mol Neurobiol 43, 3329-3341 (2023). https://doi.org/10.1007/s10571-023-01388-8.
17. Guo H, Li F, Qiu H, Xu W, Li P, Hou Y, Ding J, Chen X. Synergistically Enhanced Mucoadhesive and Penetrable Polypeptide Nanogel for Efficient Drug Delivery to Orthotopic Bladder Cancer. Research (Wash D C). 2020 Aug. 3; 2020:8970135. doi: 10.34133/2020/8970135. eCollection 2020.
18. Han, Jiansong, et al. Mechanisms of BCG in the treatment of bladder cancer-current understanding and the prospect. Biomedicine & pharmacotherapy 129 (2020): 110393.
19. Hudson M A, Brown E J, Ritchey J K, Ratliff T L. Modulation of fibronectin-mediated Bacillus Calmette-Gue'rin attachment to murine bladder mucosa by drugs influencing the coagulation pathways. Cancer Res, 1991, 51: 3726-3732.
20. Hurst R E, Moldwin R M, Mulholland S G. Bladder defense molecules, urothelial differentiation, urinary biomarkers, and interstitial cystitis. Urology, 2007, April; 69(4 Suppl): 17-23. doi: 10.1016/j.urology.2006.03.083.
21. Hurst R E. Structure, function, and pathology of proteoglycans and glycosaminoglycans in the urinary tract. Review World J Urol. 1994; 12(1):3-10. doi: 10.1007/BF00182044.
22. Kageyama Y, Kawakami S, Fujii Y, Kihara K, Oshima H. Bacillus Calmette-Guérin enhances production and secretion of type I V collagenases in peripheral blood mononuclear cells. Jpn J Cancer Res. 1997 March; 88(3):281-8. doi: 10.1111/j.1349-7006.1997.tb00379.x.
23. Kristensen S, Tian Y, Klegerman M E, Groves M J. Origins of BCG surface charge: effect of ionic strength and chemical modifications on zeta potential of Mycobacterium bovis BCG, Tice substrain, cells. Microbios. 1992; 70(284-285):185-98.
24. Lele B S, Hoffman A S. Mucoadhesive drug carriers based on complexes of poly(acrylic acid) and PEGylated drugs having hydrolysable PEG-anhydride-drug linkages. Journal of Controlled Release, vol. 69, no. 2, pp. 237-248, 2000.
25. Marengo et al. Redox homeostasis and cellular antioxidant systems: crucial players in cancer growth and therapy. Oxidative Medicine and Cellular Longevity. 2016; 2016:16. doi: 10.1155/2016/6235641.6235641.
26. Melekos M D, Chionis H, Pantazakos A, Fokaefs E, Paranychianakis G, Dauaher H. Intravesical bacillus Calmette-Gudrin immunoprophylaxis of superficial bladder cancer: results of a controlled prospective trial with modified treatment schedule. J Urol, 1993, 149: 744-748.
27. Nakamura T, Fukiage M, Higuchi M, Nakaya A, Yano I, Miyazaki J, Nishiyama H, Akaza H, Ito T, Hosokawa H, Nakayama T, Harashima H. Nanoparticulation of BCG-CWS for application to bladder cancer therapy. J Control Release. 2014 Feb. 28; 176:44-53. doi: 10.1016/j.jconrel.2013.12.027. Epub 2013 Dec. 31.
28. Pelicano et al. ROS stress in cancer cells and therapeutic implications. Drug Resistance Updates. 2004; 7(2):97-110. doi: 10.1016/j.drup.2004.01.004.
29. Petit J F, Wietzerbin J, Das B C, Lederer E. Chemical structure of the cell wall of Mycobacterium tuberculosis var. bovis, strain BCG. Z Immunitatsforsch Exp Klin Immunol. 1975 July; 149(2-4):118-25.
30. Poggi M M, Johnstone P A, Conner R J. Glycosaminoglycan content of human bladders. A method of analysis using cold-cup biopsies. Urol Oncol 2000; 5: 234-7.
31. Ratliff T L, Palmer J O, McGarr J A, Brown E J. Intravesical Bacillus Calmette-Gudrin therapy for murine bladder tumors: initiation of the response by fibronectin-mediated attachment of Bacillus Calmette-Guerin. Cancer Res, 1987, 47: 1762-1766.
32. Reshkin S J, Cardone R A, Harguindey S. Na+-H+ exchanger, pH regulation and cancer. Recent Pat Anticancer Drug Discov 2013; 8:85-99.
33. Rockwell, C. E., Morrison, D. C., & Qureshi, N. (2010). Lipid A-mediated tolerance and cancer therapy. Lipid A in cancer therapy, 81-99.
34. Charlotta Ryk, Lotta Renstr?m Koskela, Tomas Thiel, et al., Outcome after BCG treatment for urinary bladder cancer may be influenced by polymorphisms in the NOS2 and NOS3 genes, Redox Biol. (2015) 6 REDOXD1500063.
35. Schamhart D H, De Boer E C, Vleeming R, Kurth K H. Theoretical and experimental evidence on the use of glycosaminoglycans in BCG mediated immunotherapy of superficial bladder cancer. Semin Thromb Hemost, 1994, 20: 301-309.
36. Schumacker. Reactive oxygen species in cancer: a dance with the devil. Cancer Cell. 2015; 27(2):156-157. doi: 10.1016/j.ccell.2015.01.007.
37. P. F. Severino, M. Silva, M. Carrascal, et al., Oxidative damage and response to Bacillus calmette-Gudrin, in bladder cancer cells expressing sialyltransferase ST3GAL1, BMC Cancer 18 (1).
38. G. Shah, J. Zielonka, F. Chen, et al., H₂O₂ generation by BCG induces the cellular oxidative stress response required for BCGâ□™s direct effects on urothelial carcinoma tumor biology, J. Urol. 192 (4) (2014) 1238. (2018) 198.
39. 4. G. Shah, G. Zhang, F. Chen, et al., iNOS expression and NO production contribute to the direct effects of BCG on urothelial carcinoma cell biology, Urol. Oncol. 32 (1) (2014) 45 e1-45.e9.
40. Shi M, Klegerman M E, Groves M J. The effect of washing on the surface charge of Mycobacterium bovis BCG vaccine, Tice substrain. Microbios. 1989; 60(243):97-101.
41. Songbo et al. Oxidative stress injury in doxorubicin-induced cardiotoxicity. Toxicology Letters. 2019; 307: 41-48. doi: 10.1016/j.toxlet.2019.02.013.
42. Spitz, D. R., Azzam, E. I., Jian Li, J., & Gius, D. (2004). Metabolic oxidation/reduction reactions and cellular responses to ionizing radiation: a unifying concept in stress response biology. Cancer and Metastasis Reviews, 23, 311-322.
43. Tamminga et al. Role of DNA damage and epigenetic DNA methylation changes in radiation-induced genomic instability and bystander effects in germline in vivo. Current Molecular Pharmacology. 2011; 4(2):115-125. doi: 10.2174/1874467211104020115.
44. Teppema J S, de Boer E C, Steerenberg P A, van der Meijden A P. Morphological aspects of the interaction of Bacillus Calmette-Gudrin with urothelial bladder cells in vivo and in vitro: relevance for antitumor activity? Urol Res, 1992, 20: 219-228.
45. T. Thiel, C. Ryk, V. Chatzakos, et al., Secondary stimulation from Bacillus calmette Guérin induced macrophages induce nitric oxide independent cell-death in bladder cancer cells, Cancer Lett. 348 (1-2) (2014) 119-125.
46. von Palubitzki L, Wang Y, Hoffmann S, Vidal-Y-Sy S, Zobiak B, Failla A V, Schmage P, John A, Osorio-Madrazo A, Bauer A T, Schneider S W, Goycoolea F M, Gorzelanny C. Differences of the tumour cell glycocalyx affect binding of capsaicin-loaded chitosan nanocapsules. Sci Rep. 2020 Dec. 31; 10(1):22443. doi: 10.1038/s41598-020-79882-y.
47. Yu D S, Wu C L, Ping S Y, Keng C, Shen K H. Bacille Calmette-Guerin can induce cellular apoptosis of urothelial cancer directly through toll-like receptor 7 activation. Kaohsiung J Med Sci. 2015 August; 31(8):391-7. doi: 10.1016/j.kjms.2015.05.005.
48. Zhang A, Groves M J, Klegerman M E. The surface charge of cells of Mycobacterium bovis BCG vaccine, Tice substrain. Microbios. 1988; 53(216-217):191-5.

What is claimed is:

1. A composition for treating a condition associated with hyperproliferation of cells, comprising:
at least one infectious agent selected from the group consisting of bacteria, protozoa, fungi, viruses, algae and structural components thereof, wherein the at least one infectious agent has a net negative charge and is effective to treat the condition; and
at least one metal cation in an amount effective to more than offset the net negative charge of the at least one infectious agent such that the composition has a net positive charge.

2. The composition of claim 1, wherein the at least one metal cation is contained in at least one metal complex represented by formula (I): 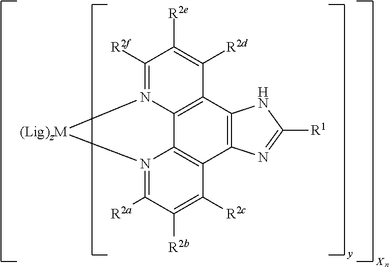
including hydrates, solvates, pharmaceutically acceptable salts and prodrugs thereof, wherein:
the at least one metal cation (M) is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
y=1,2, or 3;
z=0, 1, or 2;
Lig at each occurrence is independently selected from the group consisting of 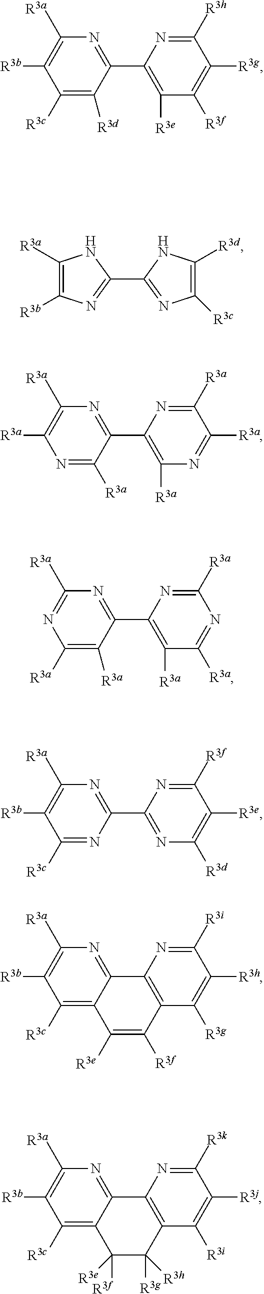 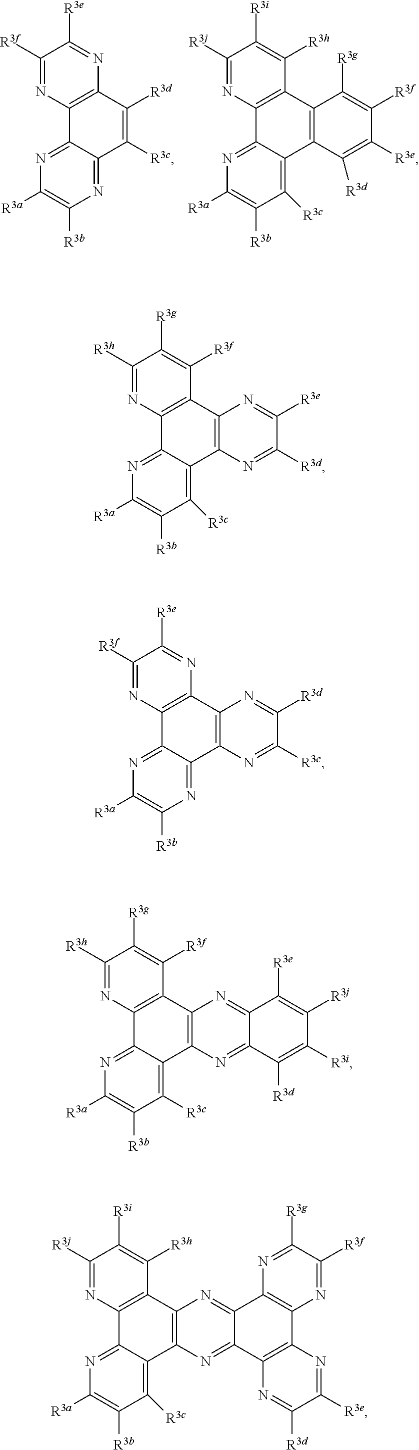 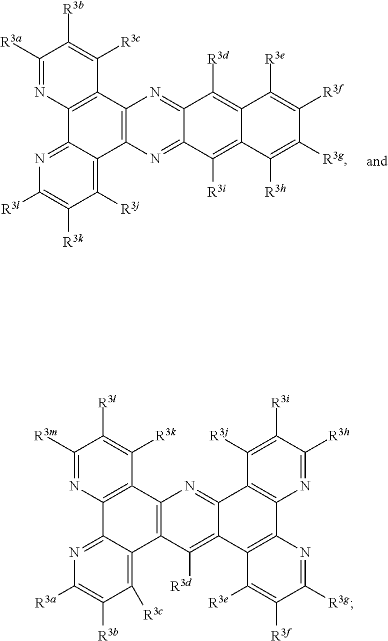
R¹ is selected from the group consisting of 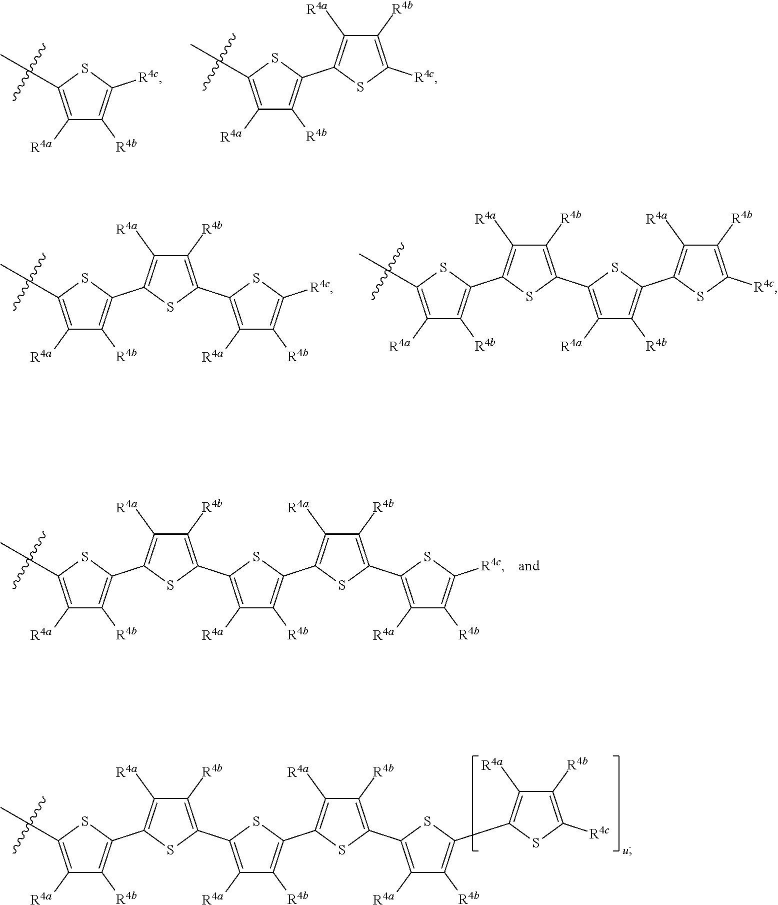
u is an integer of 1 to 20;
R^{2a}, R^{2b}, R^{2c}, R^{2d}, R^{2e}, and R^{2f} at each occurrence are each independently selected from the group consisting of hydrogen, C1-6 optionally substituted alkyl, C1-6 optionally substituted branched alkyl, C3-7 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h}, R³ⁱ, R^{3j}, R^{3k}, R^{3l}, and R^{3m} at each occurrence are each independently selected from the group consisting of hydrogen, C1-6 optionally substituted alkyl, C1-6 optionally substituted branched alkyl, C1-6 optionally substituted haloalkyl, C1-6 optionally substituted alkoxy, and CO₂R⁸;
R^{4a}, R^{4b}, and R^{4c} at each occurrence are each independently selected from the group consisting of hydrogen, C1-6 optionally substituted alkyl, C1-6 optionally substituted branched alkyl, C1-6 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;
R⁶ at each occurrence is independently selected from the group consisting of hydrogen and optionally substituted alkyl;
R⁷ at each occurrence is independently selected from the group consisting of hydrogen and optionally substituted alkyl; and
R⁸ at each occurrence is independently selected from the group consisting of hydrogen and optionally substituted alkyl.

3. The composition of claim 2, comprising complexes of the at least one infectious agent and the at least one metal complex.

4. The composition of claim 2, further comprising transferrin.

5. The composition of claim 2, wherein the at least one metal complex comprises at least one member selected from the group consisting of:
Ru(2,2′-bipyridine)₂(2-(2′,2″:5″,2′″-terthiophene)-imidazo[4,5-]A[1,10]phenanthroline);
Ru(2,2′-bipyridine)₂(2-(2′,2″:5″,2′″;5′″,2″″-quaterthiophene)-imidazo[4,5-f][1,10]phenanthroline);
Ru(4,4′-dimethyl-2,2′-bipyridine)₂(2-thiophenimidazo[4,5-f][1,10]phenanthroline);
Ru(4,4′-dimethyl-2,2′-bipyridine)₂(2-(2′,2″-bithiophene)-imidazo[4,5-f][1,10]phenanthroline);
Ru(4,4′-dimethyl-2,2′-bipyridine)₂(2-(2′,2″:5″,2′″-terthiophene)-imidazo[4,5-f][1,10]phenanthroline);
Ru(4,4′-dimethyl-2,2′-bipyridine)₂(2-(2′,2″:5″,2′″;5′″,2″″-quaterthiophene)-imidazo[4,5-f][1,10]phenanthroline);
Ru(4,4′-di-t-butyl-2,2′-bipyridine)₂(2-thiophenimidazo[4,5-][1,10]phenanthroline);
Ru(4,4′-di-t-butyl-2,2′-bipyridine)₂(2-(2′,2″-bithiophene)-imidazo[4,5-f][1,10]phenanthroline);
Ru(4,4′-di-t-butyl-2,2′-bipyridine)₂(2-(2′,2″:5″,2′″-terthiophene)-imidazo[4,5-f][1,10]phenanthroline);
Ru(4,4′-dimethoxy-2,2′-bipyridine)₂(2-(2′,2″:5″,2′″-terthiophene)-imidazo[4,5-f][1,10]phenanthroline);
Ru(5,5′-dimethyl-2,2′-bipyridine)₂(2-thiophenimidazo[4,5-][1,10]phenanthroline);
Ru(5,5′-dimethyl-2,2′-bipyridine)₂(2-(2′,2″-bithiophene)-imidazo[4,5-f][1,10]phenanthroline);
Ru(5,5′-dimethyl-2,2′-bipyridine)₂(2-(2′,2″:5″,2′″-terthiophene)-imidazo[4,5-f][1,10]phenanthroline);
Ru(6,6′-dimethyl-2,2′-bipyridine)₂(2-thiophenimidazo[4,5-][1,10]phenanthroline);
Ru(6,6′-dimethyl-2,2′-bipyridine)₂(2-(2′,2″-bithiophene)-imidazo[4,5-f][1,10]phenanthroline);
Ru(6,6′-dimethyl-2,2′-bipyridine)₂(2-(2′,2″:5″,2′″-terthiophene)-imidazo[4,5-f][1,10]phenanthroline);
Ru(4,4′-di(methylcarboxy)-2,2′-bipyridine)₂(2-(2′,2′″:5″,2′″-terthiophene)-imidazo[4,5-f][1,10]phenanthroline);
Ru(2,2′-bipyrimidine)₂(2-(2′,2″:5″,2′″-terthiophene)-imidazo[4,5-f][1,10]phenanthroline);
Ru(2,2′-bipyrimidine)(4,4′-dimethyl-2,2′-bipyridine)(2-(2′,2′″:5″,2′″-terthiophene)-imidazo[4,5-f][1,10]phenanthroline);
Ru(1,10-phenanthroline)₂(2-thiophenimidazo[4,5-f][1,10]phenanthroline);
Os(2,2′-bipyridine)₂(2-thiophenimidazo[4,5-f][1,10]phenanthroline);
Os(2,2′-bipyridine)₂(2-(2′,2″-bithiophene)-imidazo[4,5-f][1,10]phenanthroline);
Os(2,2′-bipyridine)₂(2-(2′,2″:5″,2′″-terthiophene)-imidazo[4,5-f][1,10]phenanthroline);
Os(1,10-phenanthroline)₂(2-thiophenimidazo[4,5-f][1,10]phenanthroline);
Os(1,10-phenanthroline)₂(2-(2′,2″-bithiophene)-imidazo[4,5-f][1,10]phenanthroline);
Os(1,10-phenanthroline)₂(2-(2′,2″:5″,2′″-terthiophene)-imidazo[4,5-][1,10]phenanthroline);
Os(4,4′-dimethyl-2,2′-bipyridine)₂(2-thiophenimidazo[4,5-f][1,10]phenanthroline);
Os(4,4′-dimethyl-2,2′-bipyridine)₂(2-(2′,2″-bithiophene)-imidazo[4,5-][1,10]phenanthroline);
Os(4,4′-dimethyl-2,2′-bipyridine)₂(2-(2′,2′″:5″,2′″-terthiophene)-imidazo[4,5-f][1,10]phenanthroline); and
pharmaceutically acceptable salts thereof.

6. The composition of claim 2, wherein the at least one metal complex is Ru(4,4′-dimethyl-2,2′-bipyridine)₂(2-(2′,2″:5″,2′″-terthiophene)-imidazo[4,5-f][1,10]phenanthroline) or a pharmaceutically acceptable salt thereof.

7. The composition of claim 1, wherein a single dose of the composition contains 0.001 μg to 80000 μg of the at least one metal complex and 1×10⁻⁴ to 8×10⁻⁸ CFU of the at least one infectious agent.

8. The composition of claim 1, further comprising at least one negatively charged substance additional to the at least one infectious agent, wherein the negatively charged substance is at least one of a lipopolysaccharide and a negatively charged microorganism.

9. The composition of claim 1, wherein the at least one infectious agent comprises engineered bacteria selected from the group consisting of live attenuated Mycobacterium Bovis, engineered Salmonella, engineered Staphylococcus, engineered Listeria, engineered E-coli, engineered Bifidobacterium and engineered Clostridium.

10. The composition of claim 1, comprising at least two different infectious agents selected from the group consisting of bacteria, protozoa, fungi, viruses, algae and structural components thereof.

11. A method for treating a condition associated with hyperproliferation of cells in a patient, comprising the steps:
(a) administering to the patient at least one infectious agent selected from the group consisting of bacteria, protozoa, fungi, viruses and algae, wherein the at least one infectious agent has a net negative charge and is effective to treat the condition; and
(b) administering to the patient before, during and/or after step (a) at least one metal cation,
wherein the method is effective to slow cell hyperproliferation, slow tumor growth and/or reduce tumor volume.

12. A method for treating a condition associated with hyperproliferation of cells in a patient, comprising administering to the patient a composition of claim 1 in an amount and for a duration effective to slow cell hyperproliferation, slow tumor growth and/or reduce tumor volume.

13. The method of claim 12, wherein the at least one metal cation is contained in at least one metal complex represented by formula (I): 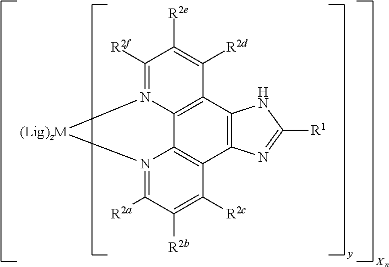
including hydrates, solvates, pharmaceutically acceptable salts and prodrugs thereof, wherein:
the at least one metal cation (M) is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
y=1,2, or 3;
z=0, 1, or 2;
Lig at each occurrence is independently selected from the group consisting of 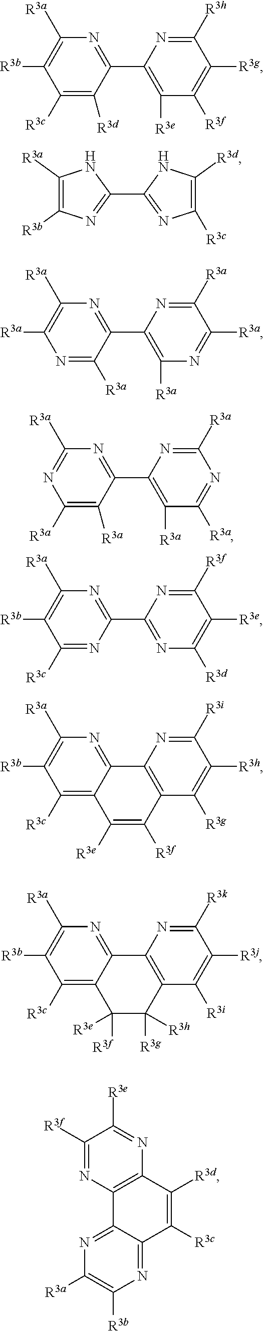 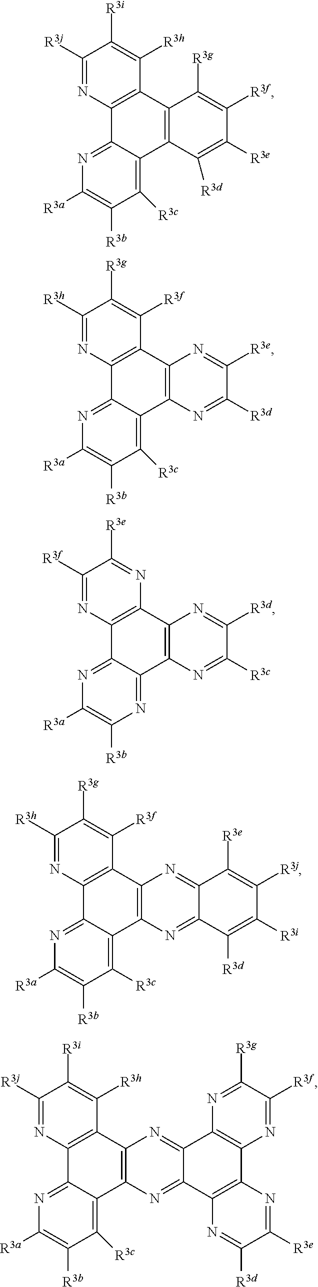 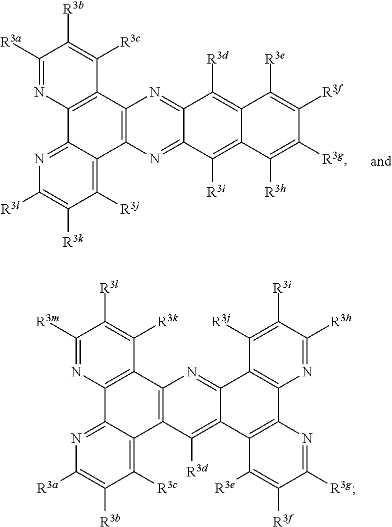
R¹ is selected from the group consisting of 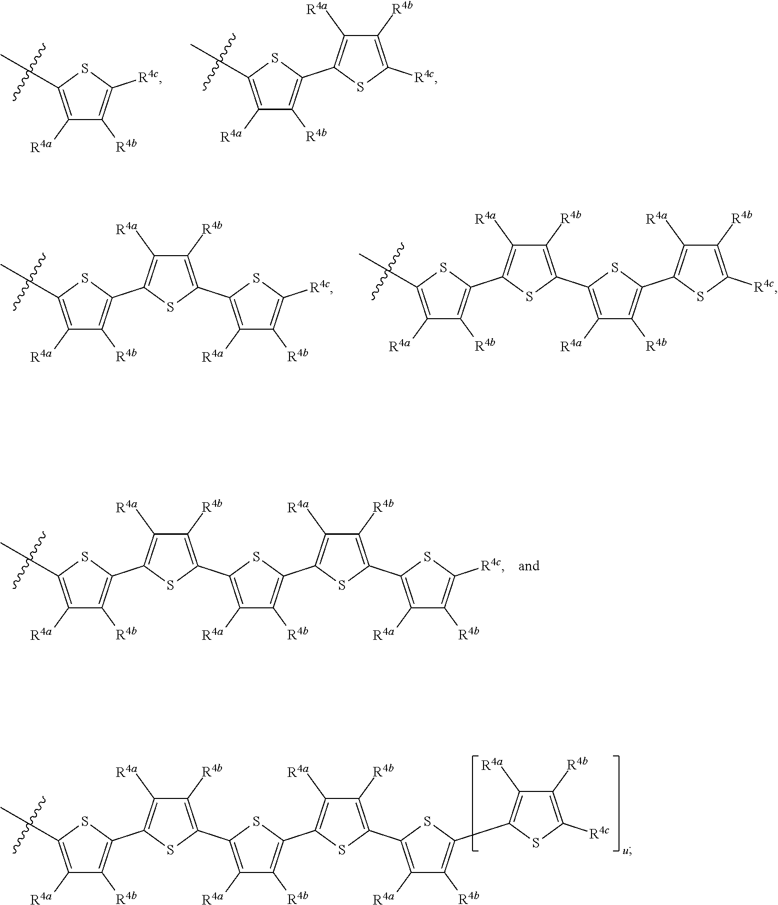
u is an integer of 1 to 20;
R^{2a}, R^{2b}, R^{2c}, R^{2d}, R^{2e}, and R^{2f} at each occurrence are each independently selected from the group consisting of hydrogen, C1-6 optionally substituted alkyl, C1-6 optionally substituted branched alkyl, C3-7 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h}, R³ⁱ, R^{3j}, R^{3k}, R^{3l}, and R^{3m} at each occurrence are each independently selected from the group consisting of hydrogen, C1-6 optionally substituted alkyl, C1-6 optionally substituted branched alkyl, C1-6 optionally substituted haloalkyl, C1-6 optionally substituted alkoxy, and CO₂R⁸;
R^{4a}, R^{4b}, and R^{4c} at each occurrence are each independently selected from the group consisting of hydrogen, C1-6 optionally substituted alkyl, C1-6 optionally substituted branched alkyl, C1-6 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;
R⁶ at each occurrence is independently selected from the group consisting of hydrogen and optionally substituted alkyl;
R⁷ at each occurrence is independently selected from the group consisting of hydrogen and optionally substituted alkyl; and
R⁸ at each occurrence is independently selected from the group consisting of hydrogen and optionally substituted alkyl.

14. The method of claim 13, wherein the composition is formed in vivo by administering the at least one metal complex and the at least one infectious agent separately.

15. The method of claim 12, wherein the composition is formed prior to being administered to the patient.

16. The method of claim 12, wherein the condition is non-muscle invasive bladder cancer.

17. The method of claim 12, further comprising administering transferrin to the patient.

18. The method of claim 13, wherein the at least one metal complex comprises at least one member selected from the group consisting of:
Ru(2,2′-bipyridine)₂(2-(2′,2″:5″,2′″-terthiophene)-imidazo[4,5-][1,10]phenanthroline);
Ru(2,2′-bipyridine)₂(2-(2′,2″:5″,2′″;5′″,2″″-quaterthiophene)-imidazo[4,5-f][1,10]phenanthroline);
Ru(4,4′-dimethyl-2,2′-bipyridine)₂(2-thiophenimidazo[4,5-f][1,10]phenanthroline);
Ru(4,4′-dimethyl-2,2′-bipyridine)₂(2-(2′,2″-bithiophene)-imidazo[4,5-f][1,10]phenanthroline);
Ru(4,4′-dimethyl-2,2′-bipyridine)₂(2-(2′,2″:5″,2′″-terthiophene)-imidazo[4,5-f][1,10]phenanthroline);
Ru(4,4′-dimethyl-2,2′-bipyridine)₂(2-(2′,2″:5″,2″″;5′″,2″″-quaterthiophene)-imidazo[4,5-f][1,10]phenanthroline);
Ru(4,4′-di-t-butyl-2,2′-bipyridine)₂(2-thiophenimidazo[4,5-][1,10]phenanthroline);
Ru(4,4′-di-t-butyl-2,2′-bipyridine)₂(2-(2′,2″-bithiophene)-imidazo[4,5-f][1,10]phenanthroline);
Ru(4,4′-di-t-butyl-2,2′-bipyridine)₂(2-(2′,2″:5″,2′″-terthiophene)-imidazo[4,5-f][1,10]phenanthroline);
Ru(4,4′-dimethoxy-2,2′-bipyridine)₂(2-(2′,2″:5″,2′″-terthiophene)-imidazo[4,5-f][1,10]phenanthroline);
Ru(5,5′-dimethyl-2,2′-bipyridine)₂(2-thiophenimidazo[4,5-][1,10]phenanthroline);
Ru(5,5′-dimethyl-2,2′-bipyridine)₂(2-(2′,2″-bithiophene)-imidazo[4,5-f][1,10]phenanthroline);
Ru(5,5′-dimethyl-2,2′-bipyridine)₂(2-(2′,2″:5″,2′″-terthiophene)-imidazo[4,5-f][1,10]phenanthroline);
Ru(6,6′-dimethyl-2,2′-bipyridine)₂(2-thiophenimidazo[4,5-][1,10]phenanthroline);
Ru(6,6′-dimethyl-2,2′-bipyridine)₂(2-(2′,2″-bithiophene)-imidazo[4,5-f][1,10]phenanthroline);
Ru(6,6′-dimethyl-2,2′-bipyridine)₂(2-(2′,2″:5″,2′″-terthiophene)-imidazo[4,5-f][1,10]phenanthroline);
Ru(4,4′-di(methylcarboxy)-2,2′-bipyridine)₂(2-(2′,2′″:5″,2′″-terthiophene)-imidazo[4,5-f][1,10]phenanthroline);
Ru(2,2′-bipyrimidine)₂(2-(2′,2″:5″,2′″-terthiophene)-imidazo[4,5-f][1,10]phenanthroline);
Ru(2,2′-bipyrimidine)(4,4′-dimethyl-2,2′-bipyridine)(2-(2′,2′″:5″,2′″-terthiophene)-imidazo[4,5-f][1,10]phenanthroline);
Ru(1,10-phenanthroline)₂(2-thiophenimidazo[4,5-f][1,10]phenanthroline);
Os(2,2′-bipyridine)₂(2-thiophenimidazo[4,5-f][1,10]phenanthroline);
Os(2,2′-bipyridine)₂(2-(2′,2″-bithiophene)-imidazo[4,5-f][1,10]phenanthroline);
Os(2,2′-bipyridine)₂(2-(2′,2″:5″,2′″-terthiophene)-imidazo[4,5-f][1,10]phenanthroline);
Os(1,10-phenanthroline)₂(2-thiophenimidazo[4,5-f][1,10]phenanthroline);
Os(1,10-phenanthroline)₂(2-(2′,2″-bithiophene)-imidazo[4,5-f][1,10]phenanthroline);
Os(1,10-phenanthroline)₂(2-(2′,2″:5″,2′″-terthiophene)-imidazo[4,5-][1,10]phenanthroline);
Os(4,4′-dimethyl-2,2′-bipyridine)₂(2-thiophenimidazo[4,5-f][1,10]phenanthroline);
Os(4,4′-dimethyl-2,2′-bipyridine)₂(2-(2′,2″-bithiophene)-imidazo[4,5-][1,10]phenanthroline);
Os(4,4′-dimethyl-2,2′-bipyridine)₂(2-(2′,2′″:5″,2′″-terthiophene)-imidazo[4,5-f][1,10]phenanthroline); and
pharmaceutically acceptable salts thereof.

19. The method of claim 13, wherein the at least one metal complex is Ru(4,4′-dimethyl-2,2′-bipyridine)₂(2-(2′,2″:5″,2′″-terthiophene)-imidazo[4,5-f][1,10]phenanthroline) or a pharmaceutically acceptable salt thereof.

20. The method of claim 12, wherein a single dose of the composition contains 0.001 μg to 80000 μg of the at least one metal cation and 1×10⁻⁴ to 8×10⁻⁸ CFU of the at least one infectious agent.

21. The method of claim 12, wherein the at least one infectious agent comprises engineered bacteria selected from the group consisting of live attenuated Mycobacterium Bovis, engineered Salmonella, engineered Staphylococcus, engineered Listeria, engineered E-coli, engineered Bifidobacterium, and engineered Clostridium.

22. The method of claim 12, further comprising administering at least one negatively charged substance additional to the at least one infectious agent, wherein the negatively charged substance is at least one of a lipopolysaccharide and a negatively charged microorganism.

23. The method of claim 12, wherein the administering step comprises administering to the patient at least two different infectious agents selected from the group consisting of bacteria, protozoa, fungi, viruses, algae and structural components thereof.
